SEQUENCE LISTING (29 sequences)

SEQ ID NO: 1 - protein, 7 aa, synthetic construct
THYPIVI

SEQ ID NO: 2 - protein, 7 aa, synthetic construct
FSRDWTS

SEQ ID NO: 3 - protein, 7 aa, synthetic construct
VNYHFPV

SEQ ID NO: 4 - protein, 7 aa, synthetic construct
TPYPIVL

SEQ ID NO: 5 - protein, 7 aa, synthetic construct
YDWTMHS

SEQ ID NO: 6 - protein, 7 aa, synthetic construct
VSYHFPV

SEQ ID NO: 7 - protein, 7 aa, synthetic construct
YKDWSEW

SEQ ID NO: 8 - protein, 7 aa, synthetic construct
DLVHYPE

SEQ ID NO: 9 - protein, 7 aa, synthetic construct
MPVARDW

SEQ ID NO: 10 - protein, 7 aa, synthetic construct
QGTDWTM

SEQ ID NO: 11 - protein, 7 aa, synthetic construct
QTSHDWL

SEQ ID NO: 12 - protein, 8 aa, synthetic construct
HAPWFRGG

SEQ ID NO: 13 - protein, 11 aa, synthetic construct
THYPIVIGGSK

SEQ ID NO: 14 - protein, 11 aa, synthetic construct
FSRDWTSGGSK

SEQ ID NO: 15 - protein, 11 aa, synthetic construct
TPYPIVLGGSK

SEQ ID NO: 16 - protein, 11 aa, synthetic construct
YDWTMHSGGSK

SEQ ID NO: 17 - protein, 11 aa, synthetic construct
VSYHFPVGGSK

SEQ ID NO: 18 - protein, 11 aa, synthetic construct
YKDWSEWGGSK

SEQ ID NO: 19 - protein, 11 aa, synthetic construct
HAPWFRGGGGS

SEQ ID NO: 20 - protein, 12 aa, synthetic construct
VIVYHFPVGGSK

SEQ ID NO: 21 - protein, 10 aa, synthetic construct
THYPIVIGGS

SEQ ID NO: 22 - protein, 10 aa, synthetic construct
FSRDWTSGGS

SEQ ID NO: 23 - protein, 10 aa, synthetic construct
TPYPIVLGGS

SEQ ID NO: 24 - protein, 10 aa, synthetic construct
YDWTMHSGGS

SEQ ID NO: 25 - protein, 10 aa, synthetic construct
VSYHFPVGGS

SEQ ID NO: 26 - protein, 10 aa, synthetic construct
YKDWSEWGGS

SEQ ID NO: 27 - protein, 10 aa, synthetic construct
HAPWFRGGGG

SEQ ID NO: 28 - protein, 11 aa, synthetic construct
VIVYHFPVGGS

SEQ ID NO: 29 - protein, 7 aa, synthetic construct
TYHIPIV

(19) United States
(12) Patent Application Publication
(10) Pub. No.: US 2026/0273112 A1
(43) Pub. Date: Sep. 17, 2026

(54) CD38 TARGETED PEPTIDES FOR CANCER IMAGING

(71) Applicants: Washington University, St. Louis, MO (US); University of Virginia Patent Foundation, Charlottesville, VA (US)

(72) Inventors: Monica Shokeen, St. Louis, MO (US); Kimberly Kelly, Charlottesville, VA (US)

(21) Appl. No.: 19/566,508

(22) Filed: Mar. 13, 2026

Related U.S. Application Data
(60) Provisional application No. 63/771,300, filed on Mar. 13, 2025.

(51) Int. Cl. A61K 51/08; C07K 7/06

(52) U.S. Cl. CPC A61K 51/088; C07K 7/06; A61K 2123/00

(57) ABSTRACT

The present teachings include compositions, methods of synthesis, and methods of use for an imaging agent that preferentially binds to CD38, including but not limited to a peptide or peptidomimetic ligand radiolabeled for use in PET imaging to identify multiple myeloma, help select treatments for multiple myeloma, and monitor relapse in multiple myeloma patients after therapy.

### CROSS-REFERENCE TO RELATED APPLICATIONS

This application claims the benefit under 35 U.S.C. § 119 (e) of U.S. Provisional Patent Application No. 63/771,300, filed Mar. 13, 2025, the entire disclosure of which is incorporated herein by reference.

### STATEMENT REGARDING FEDERALLY SPONSORED RESEARCH OR DEVELOPMENT

This invention was made with government support under CA248493 awarded by the National Institutes of Health. The government has certain rights in the invention.

### REFERENCE TO SEQUENCE LISTING

This application includes a Sequence Listing filed electronically as an XML filed named 150602134SEQ, created on Mar. 10, 2026, with a size of 25,893 bytes. The Sequence Listing in incorporated herein by reference.

### FIELD OF THE INVENTION

The present disclosure generally relates to methods and compositions for imaging multiple myeloma (MM).

### BACKGROUND OF THE INVENTION

Recent advances in the development of molecularly targeted radiopharmaceuticals have made possible the realization of precision medicine. In oncology, positron emission tomography (PET) performed with molecularly targeted radiolabeled vectors is an essential modality for patient management and improving outcomes. Development and utilization of diverse disease-specific small molecule and antibody-based probes are transforming our understanding of oncogenesis. Furthermore, there is increasing evidence of superior accuracy when nuclear imaging is synergized with liquid biopsies.

PET imaging performed with the metabolic radiopharmaceutical, [¹⁸F]fluorodeoxyglucose (FDG), has been the leading nuclear medicine tracer as demonstrated by its wide availability and frequent use. Of note, as compared to solid tumors, in hematological malignancies, FDG-PET/CT remains the mainstay for imaging of extramedullary infiltration, relapse, and assessment of inflammatory activity in leukemia as well as in FDG-avid lymphoma. Multiple myeloma is the second most common hematological cancer that causes debilitating end-organ symptoms and remains incurable. It is a disease of malignant plasma cells that originates in the bone marrow. Myeloma disorder is characterized by multiple precursor states: monoclonal gammopathy of undetermined significance and smoldering multiple myeloma. While the precursor states are not symptomatic, they are not benign either and present with a variable progression rate to overt myeloma. The unstable genome, inter-, intra- and spatial tumoral heterogeneity, age, and immunosuppressive bone marrow microenvironment all contribute to the complexity and non-uniformity of myeloma pathogenesis. Consequently, the therapy options for myeloma encompass a combination of corticosteroids, immunomodulatory agents, proteasome inhibitors, immunotherapies, and bone marrow transplantation.

Interestingly, the pathological features of multiple myeloma include the presentation of both diffuse and punctate focal lesions in the bone marrow as well as in the skeleton and visceral organs. These disease features allow for a highly informative role of PET and functional magnetic resonance imaging (MRI) in the management of patients with multiple myeloma from the initial diagnosis to therapy monitoring. Detection of minimal residual disease, relapse, and therapy response is of utmost importance in multiple myeloma patients as well. Imaging plays a forefront role in this regard. Metabolic imaging with FDG-PET has been tremendously successful in multiple myeloma patients who present with FDG avid myeloma lesions. Despite its utility, however, a known limitation of FDG-PET in multiple myeloma is the inconsistent expression of glucose transporter, GLUT-1, and hexokinase-2 enzyme in myeloma cells. FDG uptake can additionally change during the course of disease progression and the following therapy. Collectively, the inflammatory tumor microenvironment in multiple myeloma significantly influences the FDG signal to background in the bone marrow, leading to either an over-estimation or under-estimation of disease burden. Therefore, the development of new molecularly-targeted tracers for imaging multiple myeloma that can overcome the limitations described above is the logical next step. It is encouraging to witness an exciting array of new tracers in multiple myeloma targeted toward metabolism and altered proteins such as CD38, CXCR4, and BCMA.

Cluster of differentiation 38 (CD38) is overexpressed in multiple myeloma (MM) and consequently has become a valuable therapeutic target with CD38-targeted FDA approved antibodies, daratumumab and isatuximab, being used in the clinic and new antibodies undergoing clinical trials. The differential expression (low in healthy cells, high in cancerous cells) makes CD38 a valuable target for both diagnostic imaging and therapeutic intervention.

### SUMMARY OF THE INVENTION

Among the various aspects of the present disclosure is the provision of methods and compositions for imaging multiple myeloma (MM) and other CD38 related cancers.

Briefly, therefore, the present disclosure is directed to methods and compositions to specifically target CD38 in cancers such as multiple myeloma (MM).

In one aspect, compositions for an imaging agent that include a CD38 targeting peptide covalently attached to or complexed with a radionuclide binding structure are disclosed. The CD38 targeting peptide preferentially binds to CD38 epitope, and the radionuclide binding structure is configured to bind or complex to a radionuclide. In some aspects, the CD38 targeting peptide is a 5 to 15 amino acid long peptide. In some embodiments, the radionuclide binding structure includes a chelator. In some embodiments, the chelator includes one of 1,4,8,11-tetraazacyclotetradecane-1-(methane phosphonic acid)-8-(methane carboxylic acid) (TE1A1P), dodecane tetraacetic acid (DOTA), diethylenetriaminepentaacetic acid (DTPA), 1,4,8,11-tetraazacyclotetradecane-1-(methane phosphonic acid)-8-(methane carboxylic acid) (CB-TE1A1P), 1,4,7-triazacyclononane, 1-glutaric acid-4,7-acetic acid (NODAGA), 1,8-diamino-3,6,10,13,16,19-hexaazabicyclo(6,6,6) eicosane (diamine-sarcophagine) (Diamsar). In some embodiments the imaging agent includes one of CD38 peptide-11-bis(carboxymethyl)-1,4,8,11-tetraazabicyclo[6.6.2]hexadecane (CD38p-CB-TE2A), CD38 peptide-1,4,7,10-tetraazacyclododecane-N,N′,N″,N′″-tetraacetic acid (CD38p-DOTA), CD38 peptide-DOTA-polyethylene glycol (CD38p-DOTA-PEG), and CD38 peptide-1,4,7-triazacyclononane, 1-glutaric acid-4,7-acetic acid (CD38p-NODAGA), and CD38 peptide-PEG4-3,6,10,13, 16,19-hexaazabicyclo(6,6,6) eicosane (CD38p-Diamsar). In some embodiments, the radionuclide includes a radiometal. In some embodiments, the radiometal includes one of ⁶⁴Cu, ⁶⁷Cu, ²¹²Pb, ¹¹¹In, or ⁶⁸Ga. In a particular embodiment, the imaging agent is [⁶⁴Cu]Cu-CD38 peptide. In some embodiments, the imaging agent further includes a linker covalently attached to or complexed with the CD38 targeting peptide and the radionuclide binding structure; the linker being configured to attach the CD38 targeting peptide to the radionuclide binding structure. In some aspects, the CD38 targeting peptide, the radionuclide binding structure, the linker, and any combination thereof are configured to modulate the activation of CD38.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1A shows the evaluation of binding kinetics of high scoring peptides from both cluster A and cluster B.

FIG. 1B shows the evaluation of binding kinetics of chelator modified CD38 peptides.

FIG. 2A shows a radiolabeling of the ⁶⁴Cu labeled CD38-peptide NODAGA-PEG4-SL022-GGS under two conditions.

FIG. 2B shows cellular uptake and in vitro blocking of ⁶⁴Cu-NODAGA-PEG4-SL022-GGS in MM.1S-GFP—CBR-WT cells with unlabeled NODAGA-PEG4-SL022-GGS, daratumumab, and isatuximab.

FIG. 3A shows CD38 and CD31 expression in MM. 1S-GFP—CBR-WT cells.

FIG. 3B shows characterization of the disseminated MM.1S-CBR-GFP-WT tumor model with in vivo BLI showing presence of disseminated MM.1S-CBR-GFP-WT in both femurs and tibia at 5 weeks after tumor implantation.

FIG. 3C shows MM. 1S-CBR-GFP-WT tumor burden additionally evidenced in the femur via GFP intracellular expression seen with epifluorescence microscopy of unstained femur section, scale bar-20 μm.

FIG. 3D shows histological assessment of the femur with H&E staining shown at 10×, 20×, 40×, at scale of 200 μm, 100 μm, and 20 μm, respectively.

FIG. 4A shows real-time analysis of ⁶⁴Cu-NODAGA-PEG4-SL022-GGS accumulation (time-activity curve, TAC) in muscle, leg bones, spine, and pelvis for Fox Chase scid beige male mice (N=3) bearing systemically implanted MM1.S-CBR-GFP-WT xenografts.

FIG. 4B shows tissue biodistribution in tumor-bearing mice at 1 h and 4 h after tracer administration.

FIG. 4C shows tissue biodistribution at 4 h after administering ⁶⁴Cu-NODAGA-PEG4-SL022-GGS.

FIG. 4D shows BLI showing that MM. 1S-CBR-GFP-WT human MM cells injected intravenously localize to the bone marrow rich skeletal sites (spine and femurs).

FIG. 5A shows BLI demonstrating the tumor burden prior to PET.

FIG. 5B shows representative PET of MM disseminated xenografts with ⁶⁴Cu-NODAGA-PEG4-SL022-GGS.

FIG. 5C shows SUVₘₐₓ obtained from quantification of PET.

FIG. 5D shows NSG (NOD scid gamma) mice (N=10) systemically implanted with 1×10⁶ MM1.S/CBR/GFP human MM cells via lateral tail vein.

FIG. 5E shows SUVₘₐₓ obtained from quantification of PET images.

FIG. 5F shows quantification of activity as % ID/g.

FIG. 6 shows absorption and fluorescence spectra of SL022-GGS-LS288 in PBS.

FIG. 7A shows green fluorescent protein (GFP) and SL022-GGS-LS288 fluorescent images of tumor uptake at 24 h post-injection.

FIG. 7B shows SL022-GGS-LS288 tumor-to-background ratio of approximately 2.5 24 hours post-injection.

FIG. 7C shows ex vivo biodistribution data with a 8.79 tumor to muscle ratio.

FIG. 8 shows an analytical ratio-HPLC assessment of ⁶⁴Cu-NODAGA-PEG4-SL022-GGS incubated in mouse serum at 37° C. over 24 hours.

FIG. 9 shows ⁶⁴CuCl₂ radiolabeling.

FIG. 10A shows binding of ⁶⁴Cu-NODAGA-PEG4-SL022-GGS to MM.1S-CBR-GFP-WT cells either expressing (WT) or not expressing (KO) CD38, P<0.006.

FIG. 10B shows CD38 expression on the surface of MM. 1S-CBR-GFP-WT and MM. 1S-CBR-GFP-KO analyzed by flow cytometry.

FIG. 11 shows a western blot representing expression of CD38 by MM.1S-CBR-GFP-WT, MM.1S-CBR-GFP-KO, A549, and U87-MG cells.

FIG. 12 shows a representative PET comparing target compound (⁶⁴Cu-NODAGA-PEG4-SL022-GGS) with the scrambled control (⁶⁴Cu-NODAGA-PEG4-SL041-GGS) in a disseminated MM.1S-CBR-GFP-WT xenograft model.

FIG. 13A shows a representative MIP.

FIG. 13B shows axial PET obtained in a subcutaneous MM. 1S-CBR-GFP-WT human MM xenograft mouse model.

FIG. 13C shows SUVmax obtained from quantification of PET images.

### DETAILED DESCRIPTION OF THE INVENTION

Among the various aspects of the present disclosure are the provision of CD38 targeting peptide PET imaging agent compositions, methods to synthesize the disclosed compositions, and methods to identify cancer lesions using the disclosed compositions.

The present teachings include compositions, methods of synthesis, and methods of use for an imaging agent that preferentially binds to CD38, including but not limited to a peptide or peptidomimetic ligand radiolabeled for use in PET imaging to identify multiple myeloma, help select treatments for multiple myeloma, and monitor relapse in multiple myeloma patients after therapy. In some aspects, the disclosed imaging agent comprises the structure:

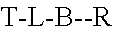

where T denotes a CD38 targeting peptide, L denotes a linker, B denotes a radionuclide binding structure, and R denotes a radionuclide used to radiolabel the imaging agent. In some aspects, the radionuclide is complexed to the remainder of the imaging agent by chelation, denoted above as a double dash (--). In some aspects, the structures are modifications of imaging agents described in Zheleznyak et al. Molecular Imaging and Biology, 2024, 26, 738-752, incorporated herein by reference in its entirety, which use CD38 peptides as T to target CD38. By way of non-limiting example, the imaging agent may be the CD38-targeted clinical-grade imaging agent, ⁶⁴Cu-NODAGA-PEG4-SL022-GGS.

In various aspects, the CD38 targeting peptide T comprises a peptide or peptidomimetic ligand configured to preferentially bind to CD38. In some aspects, the CD38 targeting peptide T is further configured to modulate the binding affinity to CD38 for optimized use in human subjects. Without being limited, any CD38 targeting peptide is thought to modulate the expression of the CD38 pathway, imparting the high contrast performance achieved in vivo, including in human subjects, as demonstrated in the examples described herein.

In some aspects, the CD38 targeting peptide T comprises a 5 to 15 amino acid long peptide. In some aspects, the CD38 targeting peptide T comprises the peptide THYPIVI (SL022; SEQ ID NO:1), FSRDWTS, (SL028; SEQ ID NO:2), VNYHFPV (SL025; SEQ ID NO:3), TPYPIVL (SEQ ID NO:4), YDWTMHS (SEQ ID NO: 5), VSYHFPV (SEQ ID NO:6), YKDWSEW (SEQ ID NO:7), DLVHYPE (SEQ ID NO:8), MPVARDW (SEQ ID NO:9), QGTDWTM (SEQ ID NO:10), QTSHDWL (SEQ ID NO:11), HAPWFRGG (SEQ ID NO:12), THYPIVIGGSK (SEQ ID NO:13), FSRDWTSGGSK (SEQ ID NO:14), TPYPIVLGGSK (SEQ ID NO: 15), YDWTMHSGGSK (SEQ ID NO:16), VSYHFPVGGSK (SEQ ID NO:17), YKDWSEWGGSK (SEQ ID NO:18), HAPWFRGGGGS (SEQ ID NO:19), VIVYHFPVGGSK (SEQ ID NO:20), THYPIVIGGS (SEQ ID NO:21), FSRDWTSGGS (SEQ ID NO:22), TPYPIVLGGS (SEQ ID NO:23), YDWTMHSGGS (SEQ ID NO:24), VSYHFPVGGS (SEQ ID NO:25), YKDWSEWGGS (SEQ ID NO:26), HAPWFRGGGG (SEQ ID NO:27), VIVYHFPVGGS (SEQ ID NO:28) or modifications thereof. In various aspects, the CD38 targeting peptide structural framework may be modified by the inclusion of various atoms or other moieties strategically positioned to improve the tumor-to-background signal within a few hours post-injection. Non-limiting examples of atoms and other moieties suitable for modification of the CD38 targeting peptide structural framework include atoms selected from fluorinated atoms, polyethylene glycols, amino acids, amino acid mimetics, organic molecules, phosphonates, sulfonates, carbohydrates, and polydentate functional groups. The modified CD38 targeting peptides are configured to bind to CD38 epitopes with high binding affinity, to clear rapidly from nontumor tissues, highlight the location of tumors, and/or determine the response of tumors to treatment.

In various aspects, the linker L may be any suitable linker moiety capable of binding together the CD38 targeting peptide T and the radionuclide binding structure B while also maintaining the targeting affinity of the CD38 targeting peptide T and the radionuclide binding properties of the radionuclide binding structure B. In some aspects, the linker L may include modifications configured to modulate the binding affinity of the imaging agent to CD38 and or the expression of the CD38 pathway, thereby optimizing the clinical performance of the imaging agent as described above.

In various aspects, the radionuclide binding structure B may be any suitable binding structure capable of binding or complexing to a radionuclide without limitation. Non-limiting examples of suitable radionuclide binding structures include chelators. Non-limiting examples of suitable chelators include chelators that coordinate metal cations such as 1,4,8,11-tetraazacyclotetradecane-1-(methane phosphonic acid)-8-(methane carboxylic acid) (TE1A1P), dodecane tetraacetic acid (DOTA), diethylenetriaminepentaacetic acid (DTPA), 1,4,8,11-tetraazacyclotetradecane-1-(methane phosphonic acid)-8-(methane carboxylic acid) (CB-TE1A1P), 1,4,7-triazacyclononane, 1-glutaric acid-4,7-acetic acid (NODAGA), and 1,8-diamino-3,6,10,13,16,19-hexaazabicyclo(6,6,6) eicosane (diamine-sarcophagine) (Diamsar).

In other aspects, the non-radioactive peptide portion (T-L-B or T-B as described below) is radiolabeled with a radionuclide (R) to form the imaging agent. In various aspects, the radionuclide may be any suitable radionuclide known to function as a radiolabel in imaging agents without limitation. In some aspects, the radionuclide may be a radiometal. Non-limiting examples of suitable radiometals include ⁶⁴Cu, ⁶⁷Cu, ²¹²Pb, ¹¹¹In, and ⁶⁸Ga. The radionuclide may be selected based on any one or more of at least several properties of the radionuclide including, but not limited to, the half-life of the radionuclide. Without being limited to any particular theory, the use of a radionuclide with a longer half-life extends the useable life of the imaging agent and facilitates the shipping of a targeting agent produced at one location to a different location for use in an imaging procedure. In one exemplary aspect, the radionuclide is ⁶⁴Cu. In another exemplary aspect, ⁶⁴Cu is coordinated to NODAGA to form the imaging agent.

In various other aspects, the CD38 targeting peptide is directly bound covalently to the radionuclide binding structure without a linker. In these other aspects, the imaging agent comprises the structure:

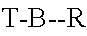

Non-limiting examples of CD38 targeting peptide modifications for use as imaging agents include CD38 peptide-11-bis(carboxymethyl)-1,4,8,11-tetraazabicyclo[6.6.2]hexadecane (CD38p-CB-TE2A), CD38 peptide-1,4,7,10-tetraazacyclododecane-N,N′,N″,N′″-tetraacetic acid (CD38p-DOTA), CD38 peptide-DOTA-polyethylene glycol (CD38p-DOTA-PEG), and CD38 peptide-1,4,7-triazacyclononane, 1-glutaric acid-4,7-acetic acid (CD38p-NODAGA), and CD38 peptide-PEG4-3,6, 10,13, 16, 19-hexaazabicyclo(6,6,6) eicosane (CD38p-Diamsar). Without being limited to any particular theory, at least some CD38 peptide modifications are designed to modulate the binding affinity of the imaging agent to CD38 in order to enhance the clinical performance of the imaging agent by providing maximum contrast and spatial imaging of bone marrow malignancies with high signal to noise characteristics.

Without being limited to any particular theory, the absolute amounts and the ratios of the molar masses of the T-L-B/T-B and R components of the disclosed compositions influence the in vivo and clinical performance of the imaging agent. In some aspects, the imaging agent contains a molar excess of the non-radioactive peptide portion (T-L-B or T-B as described above), resulting in low specific activity of the imaging agent. In various other aspects, the composition of the imaging agent comprises from about 15 μg to about 100 μg of the non-radioactive molecule (T-L-B) and from about 7 mCi to about 50 mCi of ⁶⁴Cu; compositions comprising these ranges of non-radioactive molecule and radionuclide have been proven safe and shown promise in identifying multiple myeloma in a subject, as described in the examples herein.

As described in the examples below, peptide conjugates specific for CD38 are identified, designed, synthesized, and evaluated as a viable target for MM and other pathologies. Utilizing phage display and pioneering algorithms, peptides that are selective for CD38 were identified. These selective (high scoring) peptides were synthesized using the standard solid phase peptide chemistry and were investigated for downstream in vitro and in vivo evaluations. Binding characteristics were evaluated using diverse assays. Overall, the binding data for the homology clusters was in alignment with the selectivity predictions identified by the phage display approach.

In other examples below, in vivo studies demonstrated the presence of CD38 positive cells in murine bone marrow as well as in immunofluorescence imaging of bone sections performed ex vivo resulting in the first reporting and characterization of CD38 expression in the disseminated MM.1S model.

The significant functional associations within the trans-membrane adhesion receptors of CD38 make a targeted radiolabeled peptide well-suited for imaging and therapy. While the MST assay provided accurate representation of ligand-receptor interaction, it did not consider the critical cis interactions between CD38 and the neighboring molecules in the plane of the cell membrane. Cell binding and blocking studies were used to investigate whether the cis environment affected the affinity of NODAGA-PEG4-SL022-GGS bioconjugate to the membrane expressed CD38. Interestingly, both daratumumab and isatuximab (clinically approved anti-CD38 antibodies) demonstrated efficient blocking capabilities along with the non-radioactive peptide conjugates. Although isatuximab and daratumumab do not share the binding site on CD38, it can be argued that the binding epitope for SL022 is near the antibodies' epitopes creating a strong possibility for the steric hindrance phenomenon.

In addition, as described in the examples herein, in vivo data demonstrated the targeting specificity of the CD38 peptides for the CD38 expressed on the human MM cells in the disseminated and subcutaneous xenograft models. Specificity of retention was demonstrated in diverse in vivo conditions, including the comparison with the non-specific scrambled ⁶⁴Cu-NODAGA-PEG4-SL041-GGS peptide, where the target ⁶⁴Cu-NODAGA-PEG4-SL022-GGS peptide was shown to illuminate the MM-containing bones. The ⁶⁴Cu-NODAGA-PEG4-SL022-GGS peptide accumulated in disease-bearing bones versus the bones of tumor-naïve animals. Significantly higher tracer uptake was demonstrated in tumor-bearing skeletal tissues such as spine, pelvis, and legs even though CD38 was known to be expressed at low levels in various tissues. As expected, the rapid blood clearance and excretion via kidneys was apparent.

The present disclosure is based, at least in part, on the discovery that CD38 peptide imaging agents provide contrast to multiple myeloma in human subjects. As described herein, a CD38 targeting peptide imaging agent was produced to identify multiple myeloma in patients in the studies described in detail in the Examples below.

In accordance with a further aspect, the present teachings include methods for the use of the imaging agent to identify multiple myeloma that include injection of the imaging agent followed by PET imaging.

In various additional aspects, methods to image multiple myeloma using the disclosed imaging agents are disclosed herein. The method includes injecting a patient with a clinical dose of the imaging agent and performing PET imaging to spatially identify multiple myeloma lesions based on PET signals produced by radionuclides of the imaging agent that are preferentially bound to CD38 at the CD38 targeting peptide T of the imaging agent. In one embodiment, the PET imaging occurs from about one hour after injection to about six hours after injection of the imaging agent. In some embodiments, this imaging occurs after injecting the imaging agent radiolabeled with the radionuclide ⁶⁴Cu, whose 12.7 hour half-life makes it an ideal radionuclide for this post-injection imaging window.

In another aspect, PET imaging using the disclosed imaging agent is used to select a treatment for multiple myeloma by injecting the imaging agent, performing subsequent PET imaging, and making a treatment decision based on the imaging results. In some aspects, a method to select a treatment for a multiple myeloma patient is disclosed that includes injecting a patient with a clinical dose of the imaging agent, performing PET imaging on the patient, quantifying CD38 expression in the patient from the PET images, and identifying patients who will respond to therapies based on a high/low expression stratification of patients. In one exemplary embodiment, PET imaging occurs from about one hour to about six hours after injection of the imaging agent.

In yet another aspect, PET imaging may be used to monitor relapse in multiple myeloma patients by identifying residual disease post-treatment or the appearance of recurrence over time, including recurrent refractory disease in multiple myeloma. In some aspects, a method to monitor for relapse in a multiple myeloma patient is disclosed that includes the PET imaging methods described above (injection of a clinical dose and subsequent imaging) performed serially pre-treatment, post-treatment, and at subsequent time points to monitor the change in CD38 expression over time, wherein a significant increase in CD38 expression between the post-treatment values and subsequent values is indicative of relapse. In other aspects, PET imaging occurs from about one hour to about six hours after injection of the imaging agent for each sequential PET imaging session.

In yet another embodiment, a systems biology approach is incorporated to integrate clinical data with the imaging results to better inform treatment options.

In additional aspects, methods to manufacture and package the disclosed kits are disclosed herein. These kits may be provided in non-radioactive and radioactive forms. For the non-radioactive kits, the non-radioactive peptide portion (T-L-B or T-B) described above is packaged in a vial wherein the radionuclide can be easily added to the sample for simple radiolabeling of the imaging agent for subsequent use in a human subject in a clinical setting. For the radioactive kit form, the radiolabeled imaging agent (T-L-B-R or T-B-R) is packaged in vials in a composition configured to be directly withdrawn from the vial and injected into human subjects.

### CD38 Modulation Agents

As described herein, CD38 expression has been implicated in various diseases, disorders, and conditions. As such, modulation of CD38 (e.g., modulation of CD38) can be used for the treatment of such conditions. A CD38 modulation agent can modulate CD38 activation response or induce or inhibit CD38 receptor expression. CD38 modulation can comprise modulating the expression of CD38 on cells, modulating the quantity of cells that express CD38, or modulating the quality of the CD38 receptor on cells.

CD38 modulation agents can be any composition or method that can modulate CD38 expression on cells (e.g., increasing or decreasing CD38 receptor expression on cells in cancers). For example, a CD38 modulation agent can be an activator, an inhibitor, an agonist, or an antagonist. As another example, the CD38 modulation can be the result of gene editing.

A CD38 modulation agent can be a CD38 antibody (e.g., a monoclonal antibody to the CD38 receptor).

A CD38-modulating agent can be an agent that induces or inhibits progenitor cell differentiation into CD38-expressing cells (e.g., immune cells in the bone marrow). For example, the agent can be used to enhance or inhibit the expression of CD38 on immune cells of various types in the bone marrow.

CD38 Signal Reduction, Elimination, or Inhibition by Small Molecule Inhibitors, shRNA, siRNA, or ASOs

As described herein, a CD38 modulation agent can be used in cancer therapy. A CD38 modulation agent can be used to reduce/eliminate or enhance/increase CD38 expression and signals. For example, a CD38 modulation agent can be a small molecule inhibitor of CD38. As another example, a CD38 modulation agent can be a short hairpin RNA (shRNA). As another example, a CD38 modulation agent can be a short interfering RNA (siRNA).

As another example, RNA (e.g., long noncoding RNA (lncRNA)) can be targeted with antisense oligonucleotides (ASOs) as a therapeutic. Processes for making ASOs targeted to RNAs are well known; see e.g. Zhou et al. 2016 Methods Mol Biol. 1402:199-213. Except as otherwise noted herein, therefore, the process of the present disclosure can be carried out in accordance with such processes.

### CD38 Inhibiting Agent

One aspect of the present disclosure provides for targeting of CD38, its receptor, or its downstream signaling. The present disclosure provides methods of treating or preventing CD38-based progression in multiple myeloma based on the discovery that CD38 expression has been linked to progression in multiple myeloma.

As described herein, inhibitors of CD38 (e.g., antibodies, fusion proteins, small molecules) can reduce or prevent progression and recurrence in multiple myeloma. A CD38 inhibiting agent can be any agent that can inhibit CD38, downregulate CD38, or knockdown CD38.

As an example, a CD38 inhibiting agent can inhibit CD38 signaling.

For example, the CD38 inhibiting agent can be an anti-CD38 antibody. Furthermore, the anti-CD38 antibody can be a murine antibody, a humanized murine antibody, or a human antibody.

As another example, the CD38 inhibiting agent can be an anti-CD38 antibody, wherein the anti-CD38 antibody prevents binding of CD38 to its receptor, or prevents activation of CD38 and downstream signaling.

As an example, the anti-CD38 antibody can be Daratumumab. As another example, the anti-CD38 antibody can be Isatuximab.

As another example, the CD38 inhibiting agent can be a fusion protein. For example, the fusion protein can be a decoy receptor for CD38. Furthermore, the fusion protein can comprise a mouse or human Fc antibody domain fused to CD38.

As another example, a CD38 inhibiting agent can be an inhibitory protein that antagonizes CD38. For example, the CD38 inhibiting agent can be a viral protein, which has been shown to antagonize CD38.

As another example, a CD38 inhibiting agent can be a short hairpin RNA (shRNA) or a short interfering RNA (siRNA) targeting CD38.

As another example, a CD38 inhibiting agent can be a sgRNA targeting CD38.

Methods for preparing a CD38 inhibiting agent (e.g., an agent capable of inhibiting CD38 signaling) can comprise the construction of a protein/Ab scaffold containing the natural CD38 receptor as a CD38 neutralizing agent; developing inhibitors of the CD38 receptor “down-stream”; or developing inhibitors of the CD38 production “up-stream”.

Inhibiting CD38 can be performed by genetically modifying CD38 in a subject or genetically modifying a subject to reduce or prevent expression of the CD38 gene, such as through the use of CRISPR-Cas9 or analogous technologies, wherein, such modification reduces or prevents CD38.

### Chemical Agent:

Examples of CD38 targeting agents are described herein. CD38 targeting agents may include a formula that includes R groups that may be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein the unsubstituted phenyl ring or substituted phenyl ring can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; aryl comprising a phenyl; and heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; heterocyclyl; straight chain or branched C₁₋₁₀alkyl amine; heterocyclic amine; and aryl comprising a phenyl; and heteroaryl containing from 1 to 4 N, O, or S atoms. Any of the above can be further optionally substituted.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include —OH. The “hydroxyl” can be optionally substituted.

The terms “halogen” and “halo”, as used herein, unless otherwise indicated, include a chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, include a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C₁₋₁₀ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double-bonded to an oxygen atom and single-bonded to a hydroxyl group (—COOH). The “carboxyl” can be optionally substituted.

The term “alkenyl”, as used herein, unless otherwise indicated, can include alkyl moieties having at least one carbon-carbon double bond wherein alkyl is as defined above and including E and Z isomers of said alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above and O represents oxygen. Representative alkoxyl groups include, but are not limited to, -O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O-isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl-1-butynyl, —O-cyclopropyl, —O-cyclobutyl, —O-cyclopentyl, —O-cyclohexyl, —O-cycloheptyl, —O-cyclooctyl, —O-cyclononyl and —O-cyclodecyl, —O—CH₂-cyclopropyl, —O—CH₂-cyclobutyl, —O—CH₂-cyclopentyl, —O—CH₂-cyclohexyl, —O—CH₂-cycloheptyl, —O—CH₂-cyclooctyl, —O—CH₂-cyclononyl, —O—CH₂-cyclodecyl, —O—(CH₂)₂-cyclopropyl, —O—(CH₂)₂-cyclobutyl, —O—(CH₂)₂-cyclopentyl, —O—(CH₂)₂-cyclohexyl, —O—(CH₂)₂-cycloheptyl, —O—(CH₂)₂-cyclooctyl, —O—(CH₂)₂-cyclononyl, or —O—(CH₂)₂-cyclodecyl. An alkoxyl can be saturated, partially saturated, or unsaturated. The “alkoxyl” can be optionally substituted.

The term “cycloalkyl”, as used herein, unless otherwise indicated, can include an aromatic, non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C₃₋₁₀ cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also can include-lower alkyl-cycloalkyl, wherein lower alkyl and cycloalkyl are as defined herein. Examples of -lower alkyl-cycloalkyl groups include, but are not limited to, —CH₂-cyclopropyl, —CH₂-cyclobutyl, —CH₂-cyclopentyl, —CH₂-cyclopentadienyl, —CH₂-cyclohexyl, —CH₂-cycloheptyl, or —CH₂-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “heterocyclic” can be optionally substituted.

The term “indole”, as used herein, is an aromatic heterocyclic organic compound with the formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a-CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such a compound. Examples of solvates include compounds of the invention in combination with, for example: water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high-performance liquid chromatograph. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion, or other counterions. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce the dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for the treatment of the disease, disorder, or condition.

### Therapeutic Methods

Also provided is a process of treating, preventing, or reversing multiple myeloma and metastases in the bone marrow from other cancers like breast, prostate, and lung cancers, in a subject in need of administration of a therapeutically effective amount of a multiple myeloma therapy, so as to inhibit progression, decrease recurrence, and/or extend survival.

Methods described herein are generally performed on a subject in need thereof. A subject in need of the therapeutic methods described herein can be a subject having, diagnosed with, suspected of having, or at risk for developing multiple myeloma. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of therapeutic drug that treats multiple myeloma is, for example, an amount that would cause the desired therapeutic effect in a subject while minimizing undesired side effects. In various embodiments, an effective amount of a drug that treats multiple myeloma, which include but are not limited to CD38 inhibitors, described herein can substantially inhibit the appearance or recurrence of multiple myeloma, slow progression, or limit the development of drug-resistant recurrence.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of drug that treats multiple myeloma can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to inhibit CD38 expression.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

The toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from the compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treatment can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a drug that treats multiple myeloma can occur as a single event or over a time course of treatment. For example, the drug can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for cancers of various types, including but not limited to those multiple myeloma cancers and metastatic diseases described above.

A drug to treat multiple myeloma can be administered simultaneously or sequentially with another agent, such as an antibiotic, an anti-inflammatory, or another agent. For example, a drug can be administered simultaneously with another agent, such as an antibiotic or an anti-inflammatory. Simultaneous administration can occur through the administration of separate compositions, each containing one or more of a cancer drug, an antibiotic, an anti-inflammatory, or another agent. Simultaneous administration can occur through the administration of one composition containing two or more of a cancer drug, an antibiotic, an anti-inflammatory, or another agent. A cancer drug can be administered sequentially with an antibiotic, an anti-inflammatory, or another agent. For example, a cancer drug can be administered before or after the administration of an antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, epidural, intrathecal, ophthalmic, transdermal, buccal, and rectal.

Agents and compositions described herein can be administered in a variety of methods well-known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10:0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve the taste of the product; or improve the shelf life of the product.

### Screening

Also provided are methods for screening.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., a molecular weight of about 150 to about 350 kD) with relatively fewer features (e.g., less than about 3 hydrogen donors and/or less than about 6 hydrogen acceptors; hydrophobicity character xlogP of about-2 to about 4) (see e.g., Angewante (1999) Chemie Int. ed. Engl. 24, 3943-3948). In contrast, a drug-like compound is generally understood to have a relatively larger scaffold (e.g., a molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of compounds during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to a CD38 targeting peptide, albumin, ascorbic acid, and other stabilizing agents. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing the activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754; Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

To demonstrate the synthesis of the GMP-grade imaging agents disclosed herein and the use of the disclosed imaging agents in vitro, in vivo, the following experiments were conducted.

### Materials and Methods

### Reagents

⁶⁴Cu (t_{1/2}=12.7 h, β⁺; 17.8%, E_{β+max}=656 keV, β⁻, 38.4%, E_{β-max}=573 keV) was produced on a CS-15 biomedical cyclotron at Washington University School of Medicine (WUSM) with the average specific activity of 343 mCi/μg. Fluorenylmethyloxycarbonyl (Fmoc) amino acids and Rink Amide Resin were purchased from AAPPTec (Louisville, KY, USA). Dichloromethane (DCM), acetic acid, acetic anhydride, thioanisole, phenol, hydroxybenzotriazole (HOBt), HBTU (2-(1H-benzotriazol-1-yl)-1,1,3,3-tetra-methyluronium hexafluorophosphate), N, N-diisopropyl-ethylamine (DIEA), N-trityl-1,2-ethanediamine, phenol, thioanisol, dimethylformamide (DMF), N,N′-diisopropyl-carbodiimide (DIC), trifluoroacetic acid (TFA), iodine, methyl tert-butyl ether (MTBE) and Chelex® 100 sodium form were purchased from Sigma-Aldrich (St. Louis, MO, USA). NODAGA-tris (t-Bu ester) was purchased from Macrocyclics, Inc (Plano, TX, USA). Milli-Q water was obtained from a Millipore Q3 system. All the acetate buffers were prepared with Chelex®-treated milli-Q water. All other chemicals were purchased from Sigma Aldrich unless otherwise noted.

### Phage Display Screening and Selection of CD38-Specific Peptides

Using phage display and purified human CD38 protein (hCD38), the first generation high- and low-selectivity CD38-targeted peptides were identified for the development of CD38-targeted imaging agents (Saw et al., Protein Cell, 2019, 10, 787-807). Briefly, two rounds of phage display screening using the PHASTpep platform identified selective peptides for human CD38. CD38 (R&D, Catalog 2404-AC-010, Minneapolis, MN, U.S.A.) or negative control proteins, human CD4 (hCD4), human CD8 (hCD8), and GST-6His were adsorbed on Maxisorp plates (Nalgene, Nunc #442,404) according to NEB phage display protocols (NEB, Ipswich, MA). A 10 μL aliquot of PhD7 library (7 amino acid linear library) (2×10¹¹ phage) (NEB, #E8211S) diluted in blocking buffer (DPBS/1% bovine serum albumin (BSA)) was added to each well. After 1 h of incubation at room temperature, wells were washed five times with blocking buffer and the remaining bound phage eluted into 100 μL of glycine buffer (0.2 M glycine, 0.5 M NaCl, pH 2.2) for 9 min before immediately neutralizing with 17 μL of 1 M Tris-HCl (pH 9.2).

For analysis, sequencing of the selected phage clones was performed as described in Brinton et al., PLOS ONE, 11: e0155244. Samples sent to the UVA Biomolecular Research Core Facility were deep sequenced on an Illumina MiSeq Sequencer. FASTQ files were analyzed using PHASTpep (Brinton et al., PLOS ONE, 11: e0155244). PHASTpep analyzed files were aligned for amino acid sequence homology using the Smith-Waterman algorithm.

### Generation of CD38-Targeted Peptides

Novel CD38 peptide sequences identified using phage display were synthesized via standard Fmoc chemistry (Fields et al., Int J Pept Protein Res, 1990, 35, 161-214). Briefly, using the conjugated peptide NODAGA-PEG4-Thr-His-Tyr-Pro-Ile-Val-Ile-Gly-Gly-Ser-Lys-NH₂ (NODAGA-PEG4-SL022-GGS) as an example, the following protocol was used for all peptides synthesized and subsequent bioconjugation in this study. The NODAGA-PEG4-SL022-GGS peptide was prepared using a CEM Liberty Blue microwave peptide synthesizer (Matthews, NC, USA) on Rink Amide resin. The resin (0.1 mmol) was swelled in DCM for 1 h before use. Fmoc amino acids (0.5 mmol, 5 eq), coupling reagent (HBTU, 0.5 mmol, 5 eq), and DIEA (1 mmol, 10 eq) were added to the resin and the mixture was reacted for 15 min under microwave irradiation (100W, 90° C.). The resin was washed three times with DMF. Deprotection of the Fmoc group was carried out by treatment of the resin with a solution of 20% piperidine/DMF for 5 min under microwave irradiation (100W, 90° C.). Subsequently, NODAGA-tris (t-Bu ester) (3 eq) was conjugated to the peptide while still on solid support in the presence of HOBT (5 eq), HBTU (5 eq), and DIEA (6 eq) in DMF to produce the NODAGA-PEG4-SL022-GGS peptidyl resin. The peptide was released from the resin using a cleavage cocktail of TFA: thioanisol: phenol: water (85:5:5:5, v/v/v/V) for 90 min at room temperature. The cleaved peptide product was concentrated in vacuo before performing reverse-phase HPLC purification (Gilson, Middleton, WI, USA). The final product's NODAGA-PEG4-SL022-GGS (1775 Da) molecular weight was confirmed via electrospray ionization mass spectrometry with peaks observed at 889 (M+2/2) and 593 (M+3/3). All other peptide conjugates were synthesized, purified, and characterized in an equivalent manner.

Radiolabeling of NODAGA-PEG4-SL022-GGS with the Positron Emitter ⁶⁴Cu

⁶⁴Cu chloride (⁶⁴CuCl₂) (5-10 μL in 0.5 M HCl) was diluted with 0.1 M ammonium acetate buffer (pH 5.5, 50-100 μL). NODAGA-PEG4-SL022-GGS (20 μg, 11.3 nmol) was dissolved in 200 μL ammonium acetate buffer (pH 5.5) to which buffered ⁶⁴Cu solution (37 MBq, 1 mCi) was subsequently added. The reaction mixture was agitated during incubation at 70° C. for 15 min with slight shaking. The radiochemical purity (RCP) of the ⁶⁴Cu-labeled NODAGA-PEG4-SL022-GGS (⁶⁴Cu-NODAGA-PEG4-SL022-GGS) was confirmed by analytical radio-reversed phase high performance liquid chromatography (HPLC). Radiochemical reaction progress and purity were monitored using analytical radio-HPLC performed on an Agilent 1260 Infinity (Agilent Technologies, Santa Clara, CA) with a LabLogic Flow-Ram Radio HPLC detector (LabLogic Systems, Sheffield, UK). Finally, a Kinetex 5 μm XB—C18 100A 150×4.6 mm LC column was used, and the peptide was eluted with a gradient from 95:5 0.1% TFA in water: 0.1% TFA in CH₃CN to 10:90 0.1% TFA in Water: 0.1% TFA in CH₃CN over the course of 10 min. Radioactive samples were counted using a Beckman 8000 automated well-type gamma-counter (Beckman Coulter, Franklin Lakes, NJ).

### Generation of Human MM.1S CD38 Knockout Cells

CD38 knockout (KO) cells were generated by the Genome Engineering & Stem Cell Center (GESC@MGI) at Washington University in St. Louis. Briefly, synthetic gRNA targeting the sequence 5′-CATCCTGAGATGAGGTGGGTNGG was purchased from IDT, complexed with Cas9 recombinant protein, and transfected into the MM. 1S-CBR-GFP cells. Transfected cells were sorted into 96-well plates, and clones were identified using NGS to analyze out-of-frame indels for KO.

### CD38 Expressing and CD38 Knockout Human Myeloma MM.1S Cell Line Culture

Human myeloma MM.1S cells were initially obtained from American Type Culture Collection (ATCC, Manassas, VA). MM.1S cells were modified to express Click Beetle Red (CBR) luciferase and Green Fluorescent Protein (GFP) (MM.1S-CBR-GFP) by the DiPersio laboratory (Professor John F. DiPersio, Department of Medicine, WUSM, St Louis, USA) in 2014. The expression of CD38 on the MM. 1S-CBR-GFP cells (MM.1S-CBR-GFP-WT) was previously confirmed with ex vivo flow cytometry. Cells tested negative for mycoplasma and were analyzed by the Washington University Genome Engineering and induced Pluripotent Stem Cell Core via MycoAlert PLUS Mycoplasma Detection Kit (Lonza, Visp, Switzerland) in 2018 and 2021, and via MycoStrip Mycoplasma Detection Kit (InvivoGen, San Diego, CA) in 2022. Cell lines were passaged 4-5 times after thawing before use in in vitro and in vivo studies. Cells were routinely cultured in Roswell Park Memorial Institute (RPMI) 1640 medium (ThermoFisher Scientific, Waltham, MA) supplemented with 10% heat inactivated fetal bovine serum (FBS) (Sigma-Aldrich, St. Louis, MO) and 1% penicillin/streptomycin (Gibco, Pittsburgh, PA) at 37° C. with 5% CO₂ in a humidified environment. CD38-knockout human myeloma MM.1S clones (MM.1S-CBR-GFP-KO) were developed and validated by the Genome Engineering and Stem Cell Center (GESC) at WUSM. Cell culture conditions for the MM.1S-CBR-GFP-KO cell line were consistent with those of MM.1S-CBR-GFP-WT cell line.

### Western Immunoblot Assays

Cells were mechanically dissociated from tissue culture flasks using rubber-tipped cell scrapers (Sarstedt, Newton, NC) and washed three times with DPBS. Cells were incubated with 25 mM Tris-HCl PH 7.6, 150 mM NaCl, 1% NP-40, 1% sodium deoxycholate, 0.1% Sodium Dodecyl Sulfate (SDS) buffer (RIPA lysis buffer, Abcam, Cam-bridge, MA) supplemented with EDTA-free (ethylenediaminetetraacetic acid disodium salt hydrate) protease inhibitor cocktail (Pierce, ThermoFisher, Waltham, MA) for 60 min at 4° C. followed by centrifugation at 12,000×g for 10 min. Protein content was quantified using the bicin-choninic acid protein assay (BCA, Pierce, ThermoFisher Scientific, Waltham, MA). Lysates (10-15 μg total protein) were denatured for 5 min at 100° C. and proteins were separated by non-reducing sodium dodecyl sulfate polyacrylamide gel electrophoresis (SDS-PAGE) at 100 V for 1.5 h using Mini-PROTEAN TG precast gels with Tris/Glycine/SDS buffer system (Bio-Rad, Hercules, CA). Proteins were transferred to a polyvinylidene fluoride membrane (PVDF, Millipore, Burlington, MA) at 50 V for 2 h. After blocking for 1 h in Tris buffered saline solution supplemented with 0.05% Tween-20 and 3% BSA (TBST blocking buffer, 10 mM Tris-HCl, 150 mM NaCl, and 0.05% [v/v] Tween-20, pH 7.5, 5% (w/v) non-fat milk or 3% BSA (w/v)), the PVDF membrane was rinsed once with TBST and incubated overnight at 4° C. with 1° antibody diluted in TBST blocking buffer. After two 5-min washes, the membrane was incubated with horseradish peroxidase (HRP)-conjugated secondary antibodies in TBST blocking buffer for 1 h at room temperature. Unbound secondary antibodies were removed with three washes in TBST, 10 min each. Bound secondary antibodies were detected using SuperSignal™ West Pico Plus Detection Substrate (ThermoFischer Scientific, Waltham, MA) and ChemiDoc™ Imager (Bio-Rad, Hercules, CA). Primary antibodies used for immunoblotting were rabbit monoclonal anti-CD38 (1:1000, unconjugated, clone EPR4106, Abcam, Waltham, MA) and rabbit monoclonal anti-β-Actin (1:1000, unconjugated, clone 13ES, Cell Signaling Technologies, Boston, MA). The secondary antibody used in this study was anti-rabbit IgG-HRP (1:10,000, conjugated to HRP, sc-2357, Santa Cruz Biotechnology, Dallas, TX).

### Microscale Thermophoresis

Microscale thermophoresis (MST) assays determined the binding affinity of synthesized peptides to purified CD38 protein and was performed using the Monolith NT.115 (Nanotemper Technologies, Munich, Germany). Respective peptide stock samples were diluted in MST-1 buffer (50 mM Tris-HCl, pH 7.4; 150 mM NaCl; 10 mM MgCl₂; 2% DMSO; 0.1% Tween-20; 4% SDS, 4 mM Dithiothreitol) at concentrations ranging from 0.0305 nM to 1000 nM or from 0.0305 pM to 1000 pM. Recombinant human CD38 protein (R&D, Catalog 2404-AC-010, Minneapolis, MN) was labeled with NT647 (Protein labeling kit red-maleimide, Nanotemper Technologies, Germany) and subsequently diluted to 5 nM in MST-2 buffer (50 mM Tris-HCl, pH 7.4, 150 mM NaCl, 10 mM MgCl₂, 2% DMSO, and 0.1% Tween-20). Fluorescence was assessed in MST-2 buffer. Samples were loaded into NT.115 standard capillaries, and analysis was performed at 37° C., 10% LED power, and 40% MST power. The K_{d} values were calculated from fragment concentration-dependent changes in normalized fluorescence (Fraction Bound) of different CD38 peptides based on the law of mass action using the MO Affinity Analysis v2.3 software.

### Flow Cytometry

MM.1S-CBR-GFP-WT and MM. 1S-CBR-GFP-KO cells were suspended in running buffer (DPBS with 2 mM EDTA and 0.5% BSA). Cell suspension was incubated with fluorochrome-conjugated monoclonal antibody PE/Cy7 CD38 (HIT2; BioLegend, San Diego, CA) in the dark at room temperature for 20 min. Cells were washed once with running buffer, and 7-amino-actinomycin D (7-AAD) was added to stain dead cells. Samples were run on an Attune Cytometer (ThermoFisher Scientific, Waltham, MA) and analyzed using FlowJo V10 (Tree Star, Ashland, OR), and dead cells excluded by gating out 7-AAD positive cells.

### Cell Binding Studies

To evaluate the specificity of the CD38 binding peptide, MM.1S-CBR-GFP-WT cells were suspended in binding buffer (Hank's Balanced Salt Solution with 1 mM Ca²⁺ and 1 mM Mg²⁺ (HBSS +/+, Gibco, ThermoFisher, Waltham, MA) supplemented with 0.1% BSA) at 4×10⁵ cells per 250 μL, representing the reaction volume. The cells were incubated with 300 nM ⁶⁴Cu-NODAGA-PEG4-SL022-GGS (3.3 MBq/nmol) in the presence of 75 pM unlabeled NODAGA-PEG4-SL022-GGS, 75 pM daratumumab, or 75 pM isatuximab at 4° C. for 60 min. The unbound ⁶⁴Cu-NODAGA-PEG4-SL022-GGS was removed by three washes with HBSS +/+ and cell associated activity was determined with WIZARD2® 2480 Automatic Gamma Counter (Perkin Elmer, Waltham, MA).

### Animal Models

All animal studies were performed in accordance with the WUSM Institutional Animal and Use Committee guidelines. Mice were anesthetized for all injections, treatments, and imaging with 2% isoflurane/100% O₂. Ten 5- to 6-week-old Fox Chase Severe Combined Immunodeficient (scid) beige (Charles Rivers Laboratories, Wilmington, MA, USA) or NOD scid gamma (NSG) (The Jackson Laboratory, Bar Harbor, ME, USA) mice were injected with 1×10⁶ MM.1S-CBR-GFP-WT cells, described above, in 100 μL DPBS subcutaneously or intravenously via lateral tail vein. Five additional 5- to 6-week-old NSG or Fox Chase scid beige tumor-naive mice served as non-tumor imaging and tissue biodistribution controls. Tumor burden was monitored weekly via bioluminescence imaging (BLI). Mice were randomized into cohorts for small animal PET and tissue biodistribution following administration of the radiolabeled CD38-targeted peptide. The MM. 1S-CBR-GFP-WT cell line utilized here was confirmed to have the expected CD38 expression via immunocytochemistry. Tumor burden in mouse tissues was validated in vivo with BLI and ex vivo via histology and immunohistochemistry (Greenstein et al., Exp Hematol, 2003, 31, 271-282).

### In Vivo Bioluminescence and Fluorescence Imaging

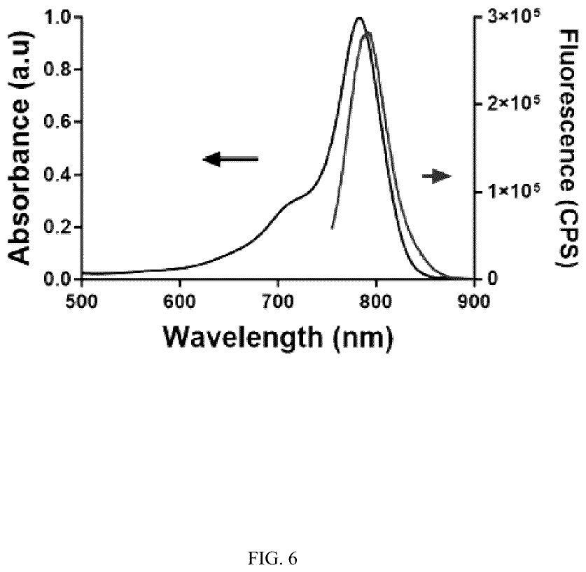

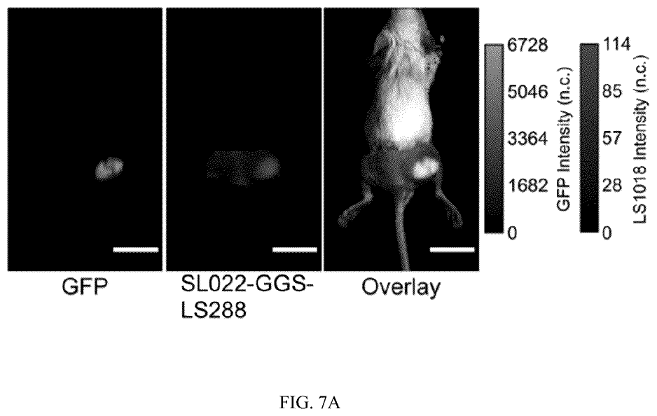

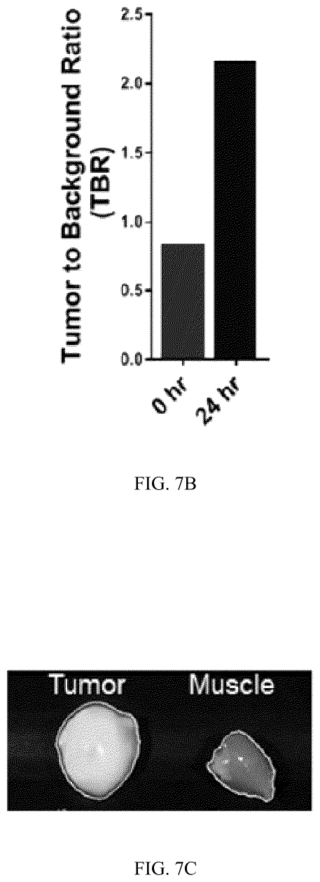

In vivo BLI was performed by the Molecular Imaging Center at Washington University in St. Louis Medical School on the days indicated on an IVIS 50 (PerkinElmer, Waltham, MA) with Living Image 4.2, a 1-300 s exposure, binning 2-8, FOV 12.5 cm, f/stop1, open filter. Mice were injected intraperitoneally with D-luciferin (150 mg/kg in DPBS, Gold Biotechnology, St. Louis, MO) and imaged under isoflurane anesthesia (2% vaporized in O₂). Total photon flux (photons/sec) was measured from fixed regions of interest (ROIs) over the entire mouse using Living Image 2.6. In vivo fluorescence imaging of GFP and SL022-GGS-LS288 (H-THYPIVI1-GGS-K(LS288)-NH₂) (FIG. 6) tumor uptake 24 h after intravenous injection was performed on an Optix MX3 time-domain diffuse optical imaging system (Advanced Research Technologies, Montreal, Canada) (FIGS. 7A-7C). Hair was removed prior to imaging with depilatory cream to improve transmission and imaged using isoflurane anesthesia (2% vaporized in O₂). The images were corrected for background to minimize auto fluorescence and diet related non-specific fluorescence.

### Histologic Assessment

After PET and biodistributions were concluded, the tibia, femurs, and spleen were harvested and fixed for 24-48 h in 10% neutral buffered formalin (NBF). Subsequent bone decalcification was performed with 14% EDTA, pH 7.4 in DPBS for 11-14 days until adequately decalcified. Specimens were processed, paraffin embedded, and sectioned at 5 μm thickness at the Musculoskeletal Histology and Morphometry Core at WUSM, Department of Orthopedic Surgery. Half of the paraffin bone and spleen sections were reserved for immunohistochemical (IHC) analysis, remaining sections were hematoxylin (Surgipath, Richmond, IL) and eosin (Richard-Allen Scientific, Kalamazoo, MI) (H&E) stained using standard procedures for routine histological analysis. Slides were viewed by a pathologist under experimentally blinded conditions.

### Immunocytochemistry

Immunocytochemistry of MM.1S-CBR-GFP-WT cells confirmed their positive CD38- and CD31-expression. 10,000 MM.1S-CBR-GFP-WT cells/mL were seeded in Falcon 8-chamber tissue culture treated glass slides (Corning, Tewksbury, MA) and incubated in Phenol-Free Dulbecco's Modified Eagle Medium (DMEM) (Gibco, ThermoFisher Scientific, Waltham, MA) supplemented with L-glutamine (Gibco, ThermoFisher Scientific, Waltham, MA) until confluent. Cells were fixed using ice cold 4% paraformaldehyde in DPBS, pH 7.4 for 10 min at room temperature, washed three times with DPBS, and permeabilized by incubating for 10 min with 0.1% Triton X-100 (Sigma-Aldrich, St. Louis, MO) in DPBS. Nonspecific binding was blocked by incubating cells with 1% BSA, 0.1% Tween 20 in DPBS for 30 min. After overnight incubation at 4° C. with anti-CD38 mouse polyclonal anti-body (#PA5-95,840, Invitrogen, ThermoFisher Scientific, Waltham, MA, 1:100 dilution) or anti-CD31 mouse monoclonal antibody (JC/70A #MA5-13, 188, Invitrogen, ThermoFisher Scientific, Waltham, MA, 1:50 dilution), cells were washed and incubated for 1 h at room temperature with Alexa Fluor 594- or 647-conjugated secondary goat anti-mouse IgG1 (cross-adsorbed, #A-21240, Invitrogen, ThermoFisher Scientific, Waltham, MA, 1:50 dilution) or F(ab′)2-goat anti-rabbit IgG (H+L) (cross adsorbed, #A48285, Invitrogen, ThermoFisher Scientific, Waltham, MA, 1:100 dilution). Cells were subsequently counter stained for nuclei visualization in 0.1 μg/mL Hoechst 33,345 (ThermoFisher Scientific, Waltham, MA) for 1 min and coverslips mounted with fluorescence preserving ProLong Glass Anti-Fade Mountant (Invitrogen, ThermoFisher Scientific, Waltham, MA).

### Immunohistochemistry of CD38 Expression

Immunohistochemistry (IHC) of the Formalin-Fixed, Paraffin Embedded (FFPE) tumor sections was performed by baking slides for 2 h at 60° C., deparaffinizing with xylenes, and rehydrating through a graded ethanol to distilled water series. Heat-induced antigen retrieval was carried out using citrate-based Antigen Unmasking Solution (Vector Laboratories, Burlingame, CA). Tissue was permeabilized and nonspecific binding blocked using 0.2% Triton X-100 (Sigma-Aldrich, St. Louis, MO) and 8% normal goat serum (Sigma-Aldrich, St. Louis, MO) in DPBS. Slides were incubated overnight at 4° C. with anti-CD31 mouse monoclonal antibody (JC/70A #MA5-13,188, Invitrogen, 1:100 dilution) or anti-CD38 rabbit monoclonal antibody (EPR4106 ab108403, Abcam, Waltham, MA, 1:500 dilution) in 3% BSA in DPBS. After washing, Alexa Fluor 647- or 594-conjugated secondary antibodies goat anti-mouse IgG1 (cross adsorbed, #A-21240, Invitrogen, ThermoFisher Scientific, Waltham, MA, 1:100 dilution) or F(ab′)₂ goat anti-rabbit IgG (H+L) (cross adsorbed, #A48285, Invitrogen, ThermoFisher Scientific, Waltham, MA, 1:100 dilution) were added to the slides and incubated for 2 h in the dark. The slides were counter-stained with Hoechst 33,342 (ThermoFisher Scientific, Waltham, MA), and sealed as in ICC protocol.

### Microscopic Analysis of ICC and IHC

Microscopy of ICC and IHC slides was performed on either Olympus BX51 epifluorescence microscope or an Olympus Fluoview 1000 confocal microscope (Olympus, Center Valley, PA). The Olympus BX51 epifluorescence microscope was equipped with 40×, 60×, 100× objectives and DAPI, GFP, Texas Red, Cy5, Cy7, and 775/845 filter sets; the con-focal microscope was equipped with 505, 560, 635, 705, and 785 nm lasers with 20×, 40×, 60× objectives. Tumor images were acquired with Olympus cellSens or Cell Standard 1.6 software and exported for analysis with ImageJ software (NIH, Bethesda, MD).

### Small Animal PET

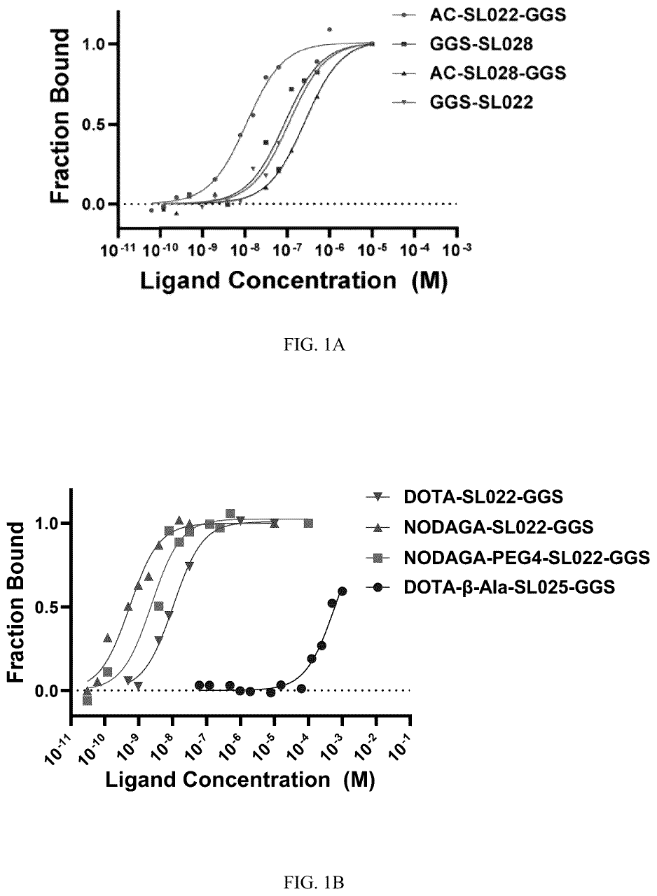

PET studies were conducted at the Preclinical Imaging Facility at the Mallinckrodt Institute of Radiology, WUSM. Animals from the Fox Chase scid beige (5-6 weeks old males, Charles River Laboratory, Wilmington, MA) or the NSG (5-6 weeks old males, NOD scid gamma, Jackson Laboratory, Bar Harbor, ME) mouse strains bearing either systemically administered MM1.S-CBR-GFP-WT xenograft, a subcutaneous MM1.S-CBR-GFP-WT xenograft, or a tumor-naive control were anaesthetized under continuous Isoflurane vapor (Pivetal®, DCM, WUSM, 2% vaporized in O₂) and injected with 6.6 MBq/nmol (FIGS. 1A and 1B) ⁶⁴Cu-NODAGA-PEG4-SL022-GGS via lateral tail vein. Imaging was performed 1 h, 2 h, or 4 h after the injection of the indicated bioconjugates using a Mediso nanoScan PET122S/CT1520 in vivo imager (Mediso, Arlington, VA) or Siemens Inveon MM PET/CT scanner (Siemens Corporation, Washington, D.C.). Mediso coincident photons were filtered with an energy window between 400 and 600 keV, and reconstruction was performed with four full iterations, six subsets per iteration with an isotropic voxel size of 0.4 mm³ using the TeraTomo 3D reconstruction algorithm. CT-based attenuation correction was applied for PET reconstruction. Siemens Inveon coincidences were filtered with energy window between 350-650 keV; the reconstruction algorithm was OSEM3D/MAP with 0.8 mm³ voxel size and CT-based attenuation correction.

### Tissue Biodistribution of ⁶⁴Cu-NODAGA-PEG4-SL022-GGS

Biodistribution studies were conducted according to previously published protocols (Boswell et al., J Med Chem, 2004, 47, 1465-1474 and Sun et al., J Med Chem, 2022, 45, 469-477). Briefly, 6.6 MBq/nmol ⁶⁴Cu-NODAGA-PEG4-SL022-GGS was injected via the tail vein. Animals were sacrificed at selected time points after, or after the conclusion of PET, organs of interest were harvested, weighed, and the associated radioactivity determined on a y-counter. After correcting for background and decay, the percent-injected dose per gram (% ID/g) was calculated by comparison to a weighed and counted standard.

### Secondary Analysis of Multiple Myeloma Research Foundation (MMRF) CoMMpass Study Data

We performed a secondary analysis of data from the MMRF CoMMpass study (Courtesy, Mark Fiala, PhD and Ravi Vij, MD). CoMMpass is a longitudinal study of over 1000 MM patients which incorporates both clinical and genomic data. As part of the CoMMpass study, RNA-sequencing (RNA-seq) on CD138-enriched bone marrow cells was performed using Illumina TruSeq RNA library kits by Translational Genomics Research Institute (TGEN). For this analysis, data were extracted from the MMRF Researcher Gateway Portal corresponding with Interim Analysis 22.

### Statistical Analysis

Data was presented as mean±SD and statistical analysis performed using GraphPad Prism Version 9.1.0 software (GraphPad Software, Inc., La Jolla, CA) and Microsoft Excel. A two-tailed unpaired t-test was used for data with two groups and one variable. Data with one variable and multiple groups were analyzed with a one-way ANOVA and Tukey's or Dunnett's multiple comparisons test to determine the adjusted P-value. Data with two variables and multiple groups were analyzed with a two-way ANOVA and Tukey's multiple comparisons test to determine the adjusted P-value. P values of less than 0.05 were considered statistically significant.

### Results:

### Phage Display Identification of CD38 Selective Peptides

To identify peptides selective for CD38, we biopanned a random, 7 amino acid peptide phage library on recombinant hCD38 as well as hCD4, hCD8, and GST-His. After two rounds of biopanning on each protein, phage were harvested, amplified, and processed for next generation sequencing (NGS) (Brinton et al., PLOS ONE, 2016, 11: e0155244). FASTQ files were uploaded into PHASTpep, translated into peptides, and tables of frequency produced. All sequences were normalized to the naïve library (preselection) sequence frequencies and finally the fold change for each peptide frequency between hCD38 and the average of the same peptide frequency from the hCD4, hCD8, and GST-His screens was calculated. The Smith-Waterman homology algorithm was employed to identify clusters of similar peptides (Table 1). From the analysis, two clusters emerged with more than 5 peptides in the homology family and also had high selectivity (PHASTpep Score, Table 1). In both clusters, the top peptide sequence had a greater than 700- and 670-fold selectivity for hCD38 over the other proteins biopanned. Therefore, we synthesized the highest scoring peptides from both clusters, A (THYPIVI, SLO22, SEQ ID NO:1) and B (FSRDWTS, SL028, SEQ ID NO: 2). Peptide sequences with less than a tenfold selectivity score are generally non-selective and thus, we used the sequence, VNYHFPV (SL025; SEQ ID NO:3), as a negative control

TABLE 1
CD38 targeted sequences identified by
phage display.

| | |
|---|---|
| Sequence | Score |

Cluster A

| | |
|---|---|
| THYPIVI | 730.1 |
| TPYPIVL | 429.7 |
| VSYHFPV | 194.4 |
| DLVHYPE | 1.5 |
| VNYHFPV | 1.2 |

Cluster B

| | |
|---|---|
| FSRDWTS | 671.1 |
| YDWTMHS | 299.7 |
| YKDWSEW | 266.4 |
| MPVARDW | 2.1 |
| QGTDWTM | 1.7 |
| QTSHDWL | 1.7 |

### Molecular Design and Synthesis

To validate the PhastPep results, we synthesized THYPIVI (SL022; SEQ ID NO: 1) from cluster A and FSRDWTS (SL028; SEQ ID NO:2) from cluster B with the addition of the tri-amino acid sequence, GGS, on the C terminus using standard FMOC chemistry. The flexible GGS linker provides structural flexibility and has been applied to connect functional peptide domains to favor interdomain interactions or movements (Chen et al., Adv Drug Deliv Rev, 2013, 65, 1357-1369). The binding affinities of GGS-SL022, AC-SL022-GGS, GGS-SL028 and AC-SL028-GGS for CD38 were compared (Table 2). The binding affinity data prompted the attachment of GGS on the amino versus the C-terminus. To enable in vivo near infrared (NIR) fluorescence imaging, a small NIR cyanine dye (LS288) was conjugated to SL022-GGS-LS288 and SL028-GGS-LS288 (Table 4). The radionuclide chelator DOTA modified peptide sequences were prepared for the cluster A high specificity sequence SL022 and low specificity sequence VNYHFPV (SL025; SEQ ID NO:3) (Table 3, DOTA-SL022-GGS, DOTA-f3-Ala-SL022-GGS and DOTA-f3-Ala-SL025-GGS). To improve the in vivo stability of ⁶⁴Cu chelation, the chelator NODAGA was used in place of DOTA for the subsequent studies (Table 4, NODAGA-SL022-GGS and NODAGA-SL025-GGS). To enhance blood circulation without compromising the binding affinity, tetra-ethylene glycol (PEG4) was incorporated into the high specificity peptide sequence to generate NODAGA-PEG4-SL022-GGS (Table 4). In addition, we prepared a PEGylated NODAGA modified cluster A peptide without GGS tag to compare the performance with NODAGA-PEG4-SL022-GGS both in vitro and in vivo (Table 4, NODAGA-PEG4-SL022).

TABLE 2
Microscale thermophoresis (MST) analysis of binding affinities
between purified CD38 protein and various CD38 peptide sequence

| | K_{d}-Mean | K_{d}-SD | Response |
|---|---|---|---|
| Compound Label | (nmol/L) | (nmol/L) | Amplitude |
| GGS-SL022 | 104.1 | 174.8 | 7.88 |
| AC-SL022-GGS | 8.57 | 2.25 | 4.91 |
| GGS-SL028 | 82.59 | 52.94 | 1.97 |
| AC-SL028-GGS | 243.46 | 88.88 | 4.64 |

TABLE 3
Bioconjugate design and characteristics.

| Compound | | | Active | | |
|---|---|---|---|---|---|
| Label | M/W | Sequence | sequence | ClogP* | Design |
| GGS- | 1170 | H-Lys-Gly-Gly-Ser-Thr-His- | THYPIVI | −2.19 | high score |
| SL022 | | Tyr-Pro-Ile-Val-Ile-NH₂ | | | cluster A |
| AC-SL022- | 1213 | AC-Thr-His-Tyr-Pro-Ile-Val- | THYPIVI | −2.23 | high score |
| GGS | | Ile-Gly-Gly-Ser-Lys-NH₂ | | | cluster A |
| SL022- | 2125 | H-Thr-His-Tyr-Pro-Ile-Val-Ile- | THYPIVI | −17.17 | high score |
| GGS- | | Gly-Gly-Ser-Lys(LS288)-NH₂ | | | cluster A, |
| LS288 | | | | | fluorescent |
| | | | | | tagged |
| GGS- | 1226 | H-Lys-Gly-Gly-Ser-Phe-Ser- | FSRDWTS | −8.93 | high score |
| SL028 | | Arg-Asp-Trp-Thr-Ser-NH₂ | | | cluster B |
| AC-SL028- | 1269 | AC-Phe-Ser-Arg-Asp-Trp- | FSRDWTS | −8.75 | high score |
| GGS | | Thr-Ser-Gly-Gly-Ser-Lys-NH₂ | | | cluster B |
| SL028- | 2181 | H-Phe-Ser-Arg-Asp-Trp-Thr- | FSRDWTS | −15.52 | high score |
| GGS- | | Ser-Gly-Gly-Ser-Lys(LS288)- | | | cluster B, |
| LS288 | | NH₂ | | | fluorescent |
| | | | | | tagged |
| DOTA- | 1556 | DOTA-Thr-His-Tyr-Pro-Ile- | THYPIVI | −6.83 | high score |
| SL022- | | Val-Ile-Gly-Gly-Ser-Lys-NH₂ | | | cluster A, |
| GGS | | | | | DOTA modified |
| DOTA-b- | 1627 | DOTA-b-Ala-Thr-His-Tyr-Pro- | THYPIVI | −7.20 | high score |
| Ala-SL022- | | Ile-Val-Ile-Gly-Gly-Ser-Lys- | | | cluster A, |
| GGS | | NH₂ | | | DOTA-b-Ala |
| | | | | | modified |
| NODAGA- | 1527 | NODAGA-Thr-His-Tyr-Pro- | THYPIVI | −5.62 | high score |
| SL022- | | Ile-Val-Ile-Gly-Gly-Ser-Lys- | | | cluster A, |
| GGS | | NH₂ | | | NODAGA |
| | | | | | modified |
| NODAGA- | 1775 | NODAGA-PEG4-Thr-His-Tyr- | THYPIVI | −6.44 | high score |
| PEG4- | | Pro-Ile-Val-Ile-Gly-Gly-Ser- | | | cluster A, |
| SL022- | | Lys-NH₂ | | | NODAGA- |
| GGS | | | | | PEG4 modified |
| DOTA-b- | 1661 | DOTA-b-Ala-Val-Asn-Tyr-His- | VNYHFPV | −8.33 | low score |
| Ala-SL025- | | Phe-Pro-Val-Gly-Gly-Ser- | | | cluster A, |
| GGS | | Lys-NH₂ | | | DOTA-b-Ala |
| | | | | | modified |
| NODAGA- | 1561 | NODAGA-Lys-Val-Asn-Tyr- | VNYHFPV | −6.85 | low score |
| SL025- | | His-Phe-Pro-Val-Gly-Gly-Ser- | | | cluster A, |
| GGS | | NH₂ | | | NODAGA |
| | | | | | modified |
| NODAGA- | 1446 | NODAGA-PEG4-Ile-Val-Ile- | THYPIVI | −3.73 | high score |
| PEG4- | | Pro-Tyr-His-Thr-NH₂ | | | cluster A, |
| SL022 | | | | | NODAGA- |
| | | | | | PEG4 modified |
| NODAGA- | 1775 | NODAGA-PEG4-Thr-Tyr-His- | TYHIPIV | −6.44 | Scrambled |
| PEG4- | | Ile-Pro-Ile-Val-Gly-Gly-Ser- | | | peptide for high |
| SL041- | | Lys-NH₂ | | | score cluster A, |
| GGS | | | | | NODAGA- |
| | | | | | PEG4 modified |

### Microscale Thermophoresis (MST) Assay to Determine Binding Affinities

Microscale Thermophoresis (MST) measures molecular interactions based on temperature gradient-dependent mobility of fluorescent biomolecules. We assessed the specificity and affinity of 8 CD38-targeted peptides for purified CD38 protein using the MST method. MST analysis showed nanomolar range K_{d} for all the high specificity peptides from clusters A and B (FIGS. 1A and 1B and Table 3). MST analysis of the interaction between CD38 peptides modified with chelators or PEG4 validated the results from the PhastPep analysis. DOTA modified SL022 has a K_{d} of 89.2±1.04 nM, NODAGA modified SL022 has a K_{d} of 0.524±0.127 nM and NODAGA-PEG modified SL022 has a K_{d} of 7.55±0.291 nM demonstrating high specificity and affinity for binding CD38 recombinant protein (FIGS. 1A and 1B and Table 4). In contrast, the predicted low specificity/affinity binding peptide SL025 had negligible binding. The K_{d} value was 605±0.697 μM for DOTA-f3-Ala-SL025-GGS (modified with DOTA and f3-Alanine), and undetermined for NODAGA-SL025-GGS (modified with NODAGA). Based on the affinity data, serum stability results, and blood circulation kinetics, NODAGA-PEG4-SL022-GGS was the best choice for subsequent in vitro and in vivo validation studies.

TABLE 4
Binding of peptides modified with chelators for radiolabeling

| Compound | Peptide | K_{d} (Mean) |
|---|---|---|
| DOTA-β-Als- | DOTA-β-Ala-Val-Aan-Tyr-Hdis-Phe-Pro- | 605 ± 0.697 nM |
| SL025-GGS | Val-Gly-Gly-Ser-Lys-NH₂ (VNYHPV) | |
| DOTA-SL022- | DOTA-Thr-His-Tyr-Pro-Be-Val-Ile-Gly- | 89.2 ± 1.04 nM |
| GGS | Gly-Ser-Lys-NH₂ (THYPIVD) | |
| NODAGA-SL022- | NODAGA-Thr-His-Tyr-Pro-Ile-Val-Ile- | 0.524 ± 0.127 nM |
| GGS | Gly-Gly-Ser-Lys-NH₂ (THYPIVD) | |
| NODAGA-PEG4- | NODAGA-PEG4-Thr-His-Tyr-Pro-Ile-Val- | 7.55 ± 0.291 nM |
| SL022-GGS | Ile-Gly-Gly-Ser-Lys-NH₂ (THYPIVI) | |
| NODAGA-SL025- | NODAGA-Lys-Val-Asn-Tyr-His-Phe-Pro- | ND |
| GGS | Val-Gly-Gly-Ser-NH₂ (VNYHEPV) | |

### Radiolabeling the CD38 Bio-Conjugates

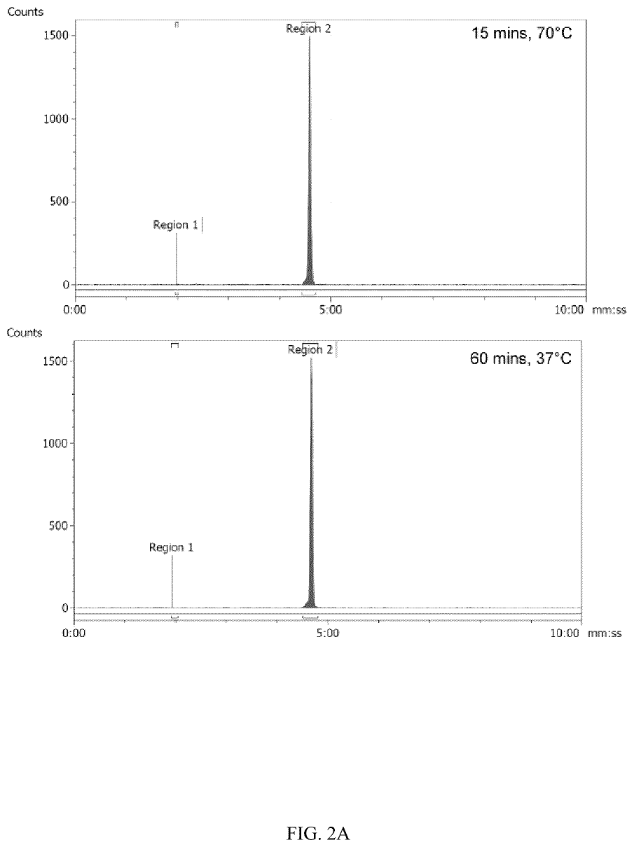

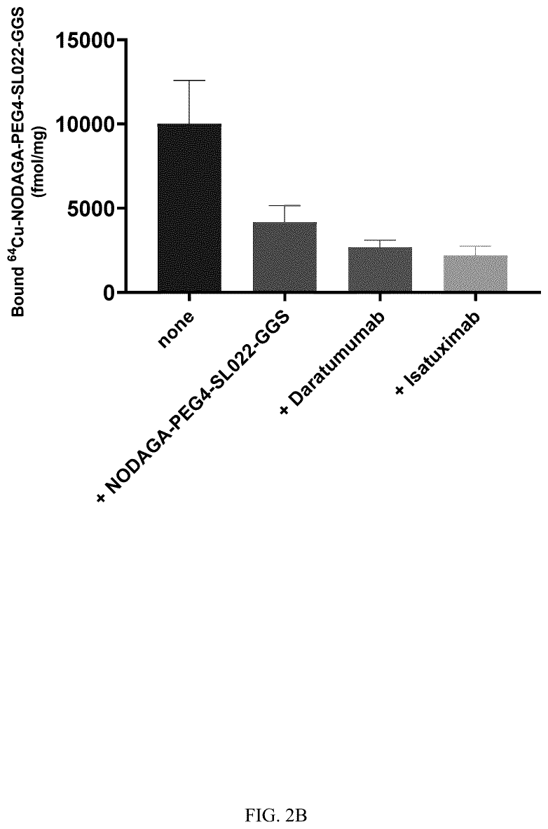

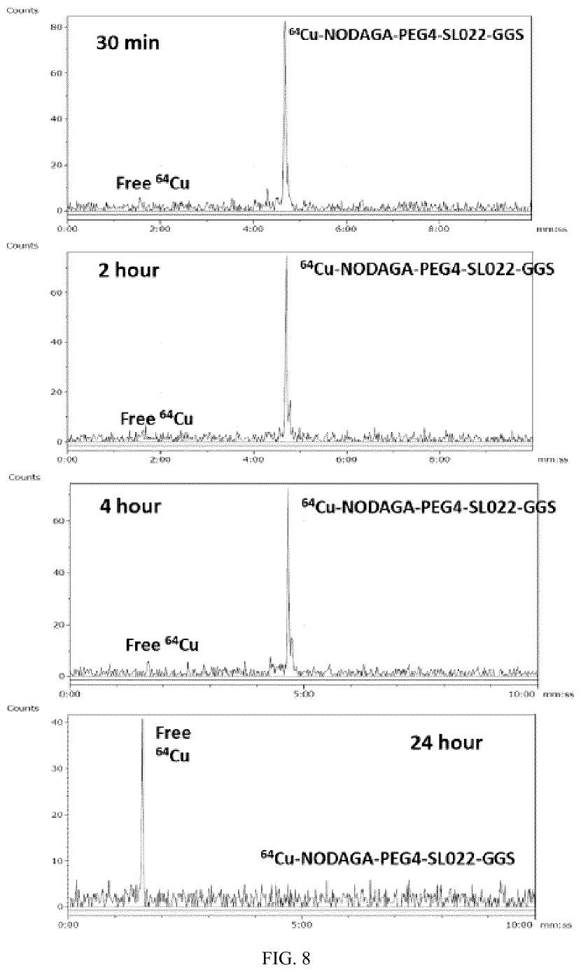

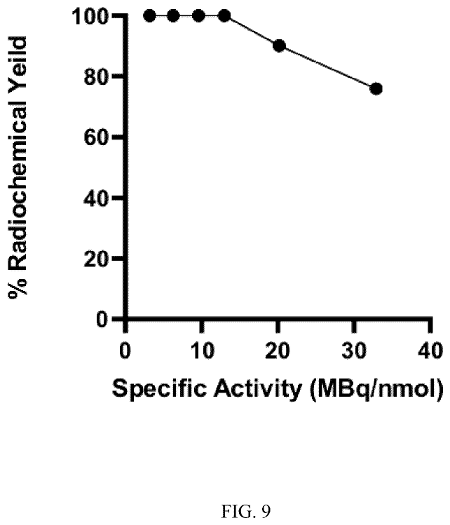

The CD38-targeted peptide bioconjugate, NODAGA-PEG4-SL022-GGS, was successfully radiolabeled with ⁶⁴Cu resulting in a specific activity (SA) of 3.3 MBq/nmol-6.6 MBq/nmol (N=10). The radiochemical purity (99±0.03%) was confirmed by analytical radio-HPLC (FIG. 2A). Serum stability studies demonstrated the robustness of ⁶⁴Cu-NODAGA-PEG4-SL022-GGS for up to 4 h (FIG. 8). The NODAGA-PEG4-SL022-GGS labeling capacity was investigated using labeling reactions with increasing amounts of ⁶⁴Cu. NODAGA-PEG4-SL022-GGS could be radiolabeled up to a maximum SA of ~148 MBq/nmol with 100% radiochemical purity (FIG. 9). Overall, the radiolabeling studies demonstrated robust labeling capacity of NODAGA-PEG4-SL022-GGS.

NODAGA-PEG4-SL022-GGS Retains its Affinity and Specificity for Binding CD38 when Expressed in its Native Cell Membrane Context

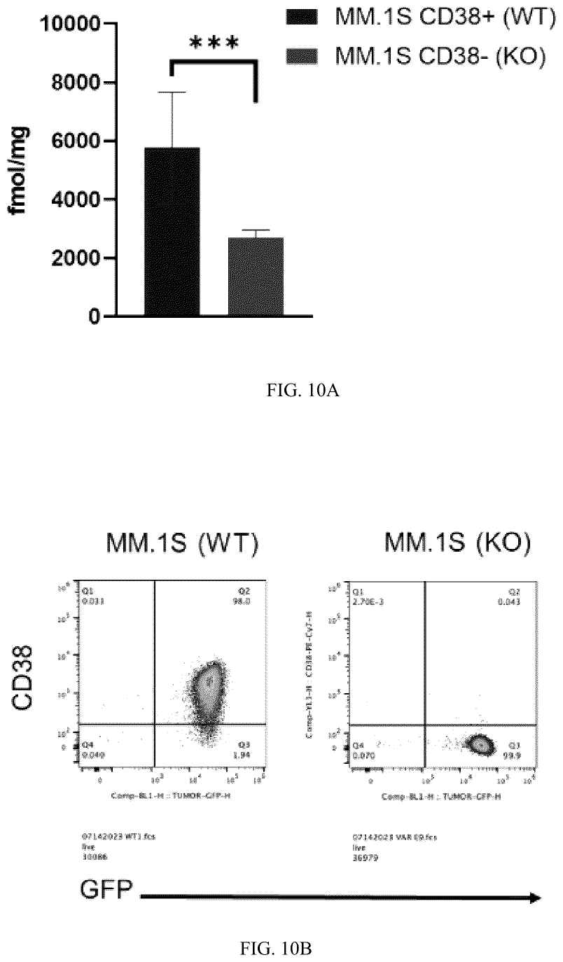

In order to determine whether the conjugates were able to identify CD38 protein expressed in its native context, we performed cell binding assays with MM.1S-CBR-GFP-WT cells. For DOTA-f3-Ala-SL025-GGS the K_{d} was calculated as 16.6 and 15.9 nM (N=2, triplicates). However, this molecule was not pursued due to the serum instability of the copper labeled molecule (⁶⁴Cu-DOTA-f3-Ala-SL025-GGS). The ability of NODAGA-PEG4-SL022-GGS to bind specifically to CD38 expressed on the surface of MM.1S-CBR-GFP-WT human MM cells was assessed in an in vitro system. NODAGA-PEG4-SL022-GGS was radiolabeled with ⁶⁴Cu with 3.3 MBq/nmol SA. MM. 1S-CBR-GFP-WT cells were allowed to bind 300 nM ⁶⁴Cu-NODAGA-PEG4-SL022-GGS for 60 min at 4° C. in the presence or absence of 75 μM of either unlabeled NODAGA-PEG4-SL022-GGS, 75 UM daratumumab, or 75 UM isatuximab. As seen in FIG. 2B, 250-fold molar excess of unlabeled NODAGA-PEG4-SL022-GGS lead to about 2-fold decrease in cell-bound ⁶⁴Cu-NODAGA-PEG4-SL022-GGS 0.4×10⁴±0.2×10⁴ fmol/mg versus 1×10⁴±0.6×10⁴ fmol/mg, P=0.029). Unlabeled daratumumab and isatuximab were also effective at preventing the binding of ⁶⁴Cu-NODAGA-PEG4-SL022-GGS to CD38 expressed by MM.1S-CBR-GFP-WT cells (0.3×10⁴±0.06×10⁴, P=0.037 for daratumumab, 0.2×10⁴±0.09×10⁴, P=0.04 for isatuximab). Furthermore, there was about 68% reduced uptake of ⁶⁴Cu-NODAGA-PEG4-SL022-GGS in the MM.1S-CBR-GFP-KO cells as compared to the MM.1S-CBR-GFP-WT cells (P<0.006) (FIG. 10A). Overall, the cell binding data showed that ⁶⁴Cu-NODAGA-PEG4-SL022-GGS bound to CD38 in a specific manner.

### Histological Characterization of MM.1S-CBR-GFP-WT In Vitro and In Vivo

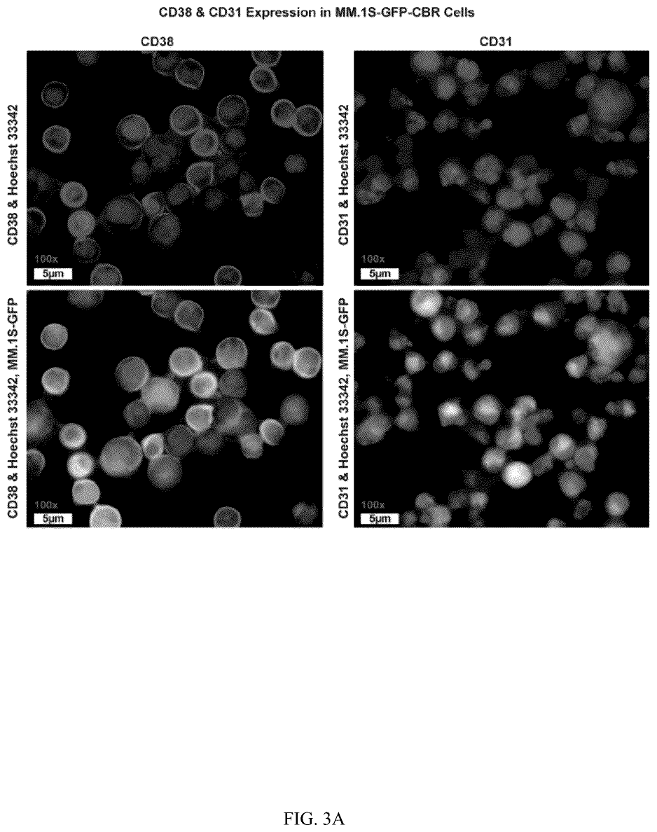

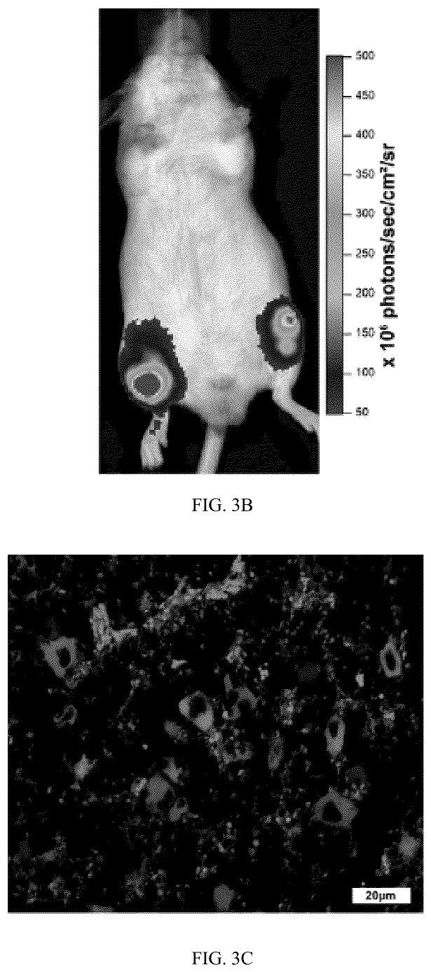

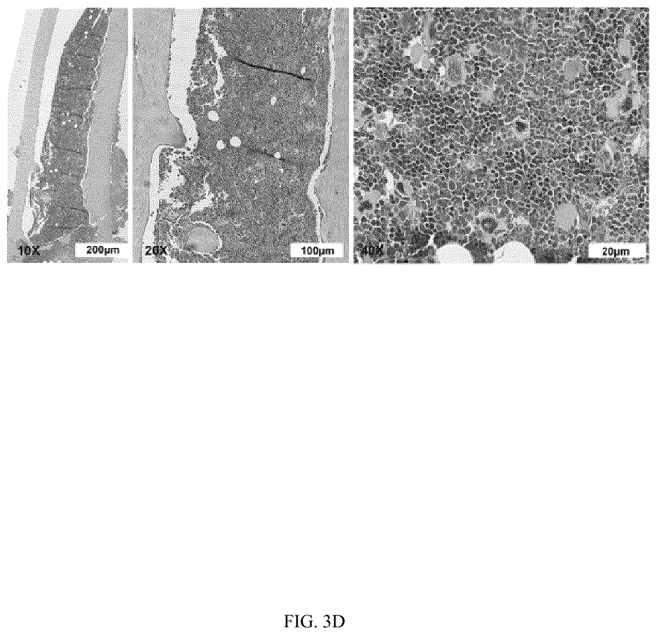

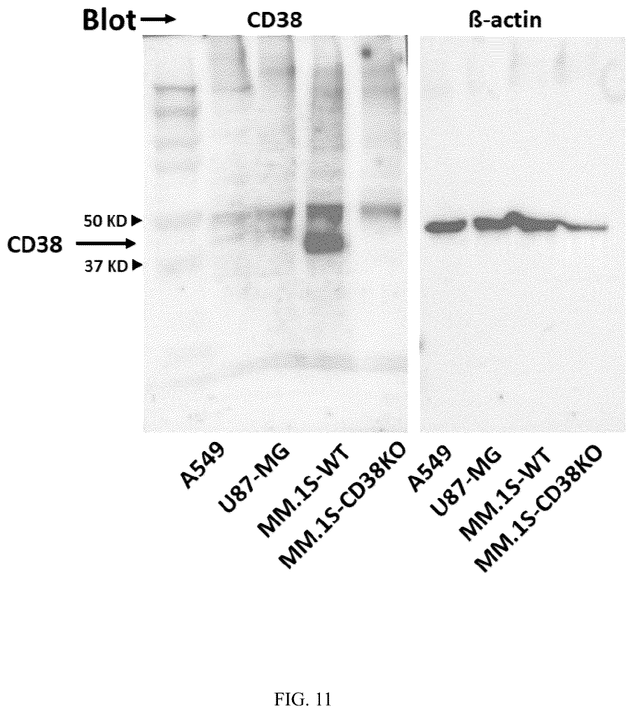

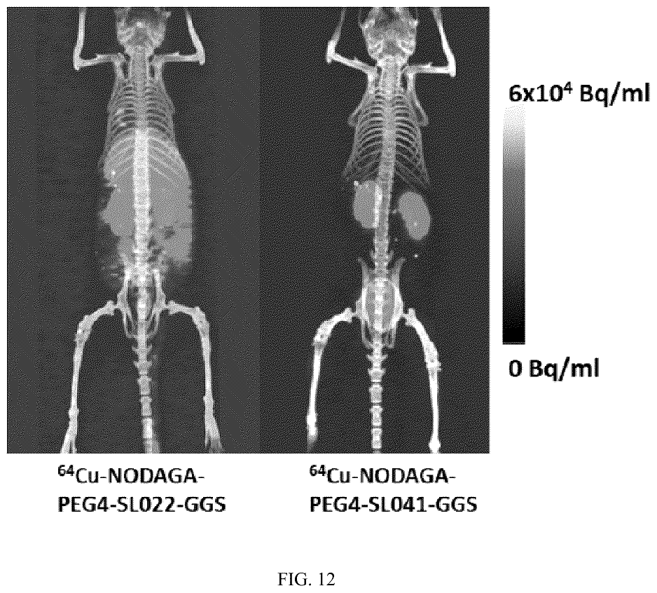

To confirm the MM.1S-CBR-GFP-WT human MM cell line as a CD38 positive MM mouse model for CD38-targeted therapies, internal GFP reporter expression within MM.1S-CBR-GFP-WT human MM cell line was demonstrated via immunocytochemistry and immunohistochemistry. MM. 1S-CBR-GFP-WT cells were incubated with anti-CD38 and anti-CD31 antibodies and imaged showing high expression of both CD38 and CD31 on the cell membrane and intra-cellularly. The membrane associated CD38 exhibited high expression on over 80% of MM.1S-CBR-GFP-WT cells (FIG. 3A). CD31 was also shown to be highly expressed. Additionally, the expression of CD38 by MM.1S-CBR-GFP-WT was examined by flow cytometry (FIG. 10B) and Western blotting (FIG. 11). These data demonstrated that CD38 expression by MM.1S-CBR-GFP-WT was higher than that of A549 cell line (human lung carcinoma) (Gao et al., Cell Death Dis, 2021, 12, 680) and U87-MG cell line (human glioblastoma) (Sun et al., Theranostics, 2019, 9, 3595-3607), both known expressors of CD38. MM.1S-CBR-GFP-KO cell line showed no expression of CD38 by either western blot or flow cytometry. The disseminated disease model was effective at establishing the disease state, as confirmed with BLI (FIG. 3B). The tumor-bearing mice were sacrificed, femur bones extracted, and immunohistochemistry performed to demonstrate in vivo presence of CD38+MM tumor burden. Unstained femur section showed GFP intracellular expression under epifluorescence microscopy indicating MM.1S-CBR-GFP-WT tumor burden in the femur (FIG. 3C). Additionally, H&E staining showed the presence of the myeloid cells while CD38 antibody staining indicated the presence of CD38+ cells (FIG. 3D).

### Tissue Biodistribution and PET

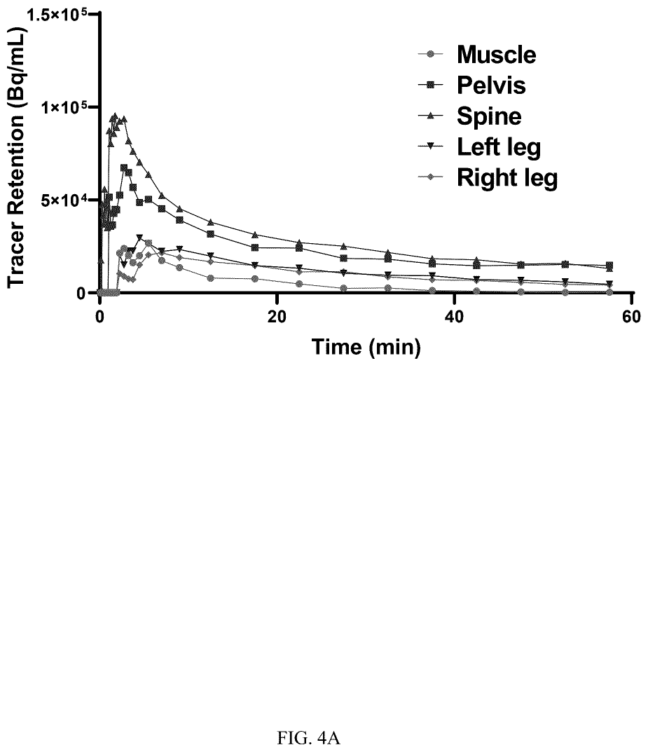

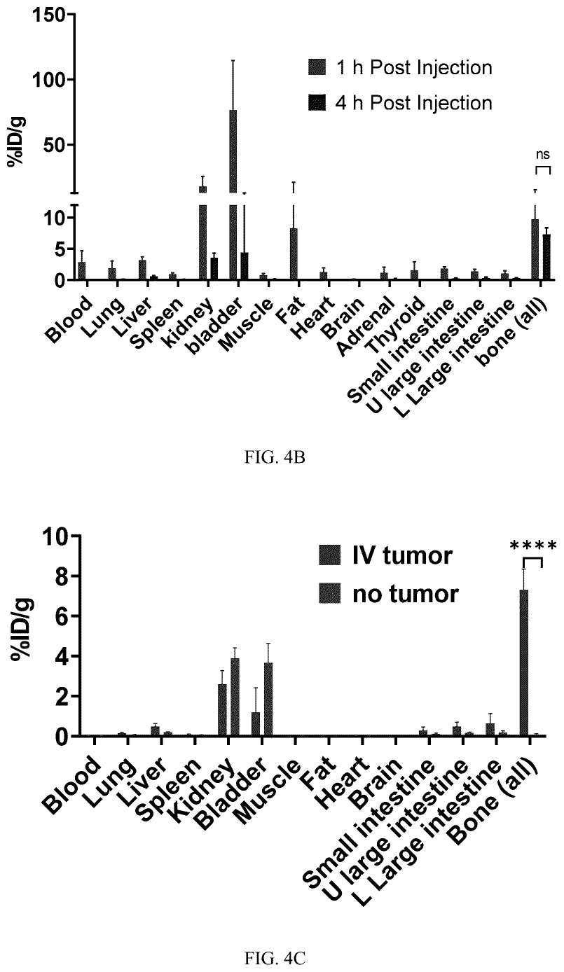

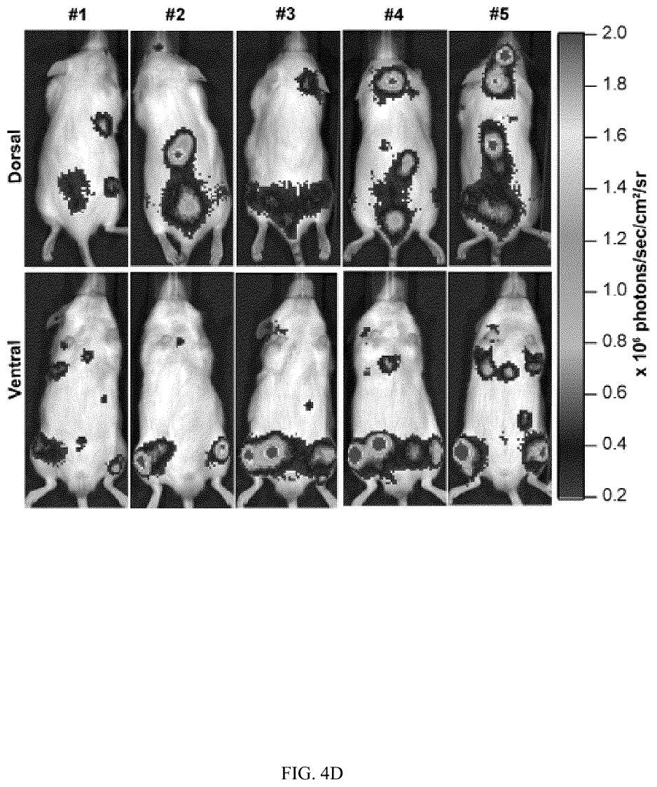

The distribution of a tracer immediately after administration is critical to understanding its in vivo behavior. Accordingly, we investigated the dynamic in vivo distribution of the tracer during the first 60 min after the administration. We established the disseminated disease in Fox Chase scid beige mice (N=3) by systemically implanting MM. 1S-CBR-GFP-WT human MM cells via lateral tail vein injection and performed the dynamic PET of ⁶⁴Cu-NODAGA-PEG4-SL022-GGS. The tissue uptake and retention were analyzed in leg, pelvis, and spine then compared to tracer retention in the muscle, a non-target tissue. The time-activity curves (TAC) representing the retention in these tissues are shown in FIG. 4D and Table 5.

TABLE 5
biodistribution of 64Cu-NODAGA-PEG4-SL022-
GGS at 1 hour and 4 hour time points

| | |
|---|---|
| 1 hour (N = 4) | 4 hour (N = 4) |

| | % ID/g | SD | % ID/g | SD |
|---|---|---|---|---|
| Blood | 2.87 | 1.80 | 0.04 | 0.03 |
| Lung | 1.90 | 1.15 | 0.12 | 0.03 |
| Liver | 3.21 | 0.49 | 0.60 | 0.14 |
| Spleen | 0.93 | 0.21 | 0.10 | 0.03 |
| kidney | 17.99 | 7.73 | 3.57 | 0.74 |
| bladder | 76.46 | 38.03 | 4.42 | 8.67 |
| Muscle | 0.77 | 0.26 | 0.07 | 0.12 |
| Fat | 8.27 | 12.84 | 0.02 | 0.02 |
| Heart | 1.23 | 0.69 | 0.06 | 0.03 |
| Brain | 0.12 | 0.04 | 0.01 | 0.00 |
| Adrenal | 1.14 | 0.91 | 0.11 | 0.17 |
| Thyroid | 1.57 | 1.34 | 0.03 | 0.02 |
| Small intestine | 1.84 | 0.28 | 0.26 | 0.07 |
| U large intestine | 1.38 | 0.33 | 0.34 | 0.15 |
| L Large intestine | 1.08 | 0.41 | 0.31 | 0.06 |
| Bone | 9.77 | 5.66 | 7.31 | 1.04 |

These data demonstrated that a significant amount of activity was detected in spine (5.05×10⁴ Bq/mL) and pelvic bones (2.27×10⁴ Bq/mL) immediately after the administration compared to the activity in muscle (0.53×10⁴ Bq/mL), P <0.0001 and P<0.0009, respectively. This significant difference in retention was maintained throughout the data acquisition. Specifically, spine retained 4.35×10⁴ Bq/mL, pelvis retained 2.18×10⁴, while muscle retained 0.47×10⁴ Bq/mL. Although the ⁶⁴Cu-NODAGA-PEG4-SL022-GGS uptake in the leg bones was not different from that in muscle during 0 to 10 min, the tracer amount was significantly higher in leg bone versus muscle from 10 min until the end of data acquisition (left leg versus muscle: 1.14×10⁴ versus 0.39×10⁴ Bq/mL, P=0.003; right leg versus muscle: 1.0×10⁴ versus 0.39×10⁴ Bq/mL, P=0.006). These data demonstrate that ⁶⁴Cu-NODAGA-PEG4-SL022-GGS targeted and retained in marrow-rich bones for the duration of the study.

As a small peptide, ⁶⁴Cu-NODAGA-PEG4-SL022-GGS was expected to be cleared from the body relatively quickly. Therefore, we compared the ex-vivo biodistribution of ⁶⁴Cu-NODAGA-PEG4-SL022-GGS at 1 h and 4 h after tracer administration. The data in FIG. 4B demonstrated rapid clearance of ⁶⁴Cu-NODAGA-PEG4-SL022-GGS from non-target organs through the kidneys 1 h after tracer administration while being retained in the target organs. Complete clearance of ⁶⁴Cu-NODAGA-PEG4-SL022-GGS from non-target organs was achieved at 4 h after tracer administration, with 80% decrease in the kidney (18±7.7% ID/g versus 3.6±0.7% ID/g) and 94% decrease in the bladder (76±38% ID/g versus 4.4±8.7% ID/g). In contrast, the tracer accumulation in bone was retained with only 25% decrease at the same 4 h time point (9.8±5.7% ID/g versus 7.3±1.04% ID/g). Importantly, the difference in bone retention between 1 h and 4 h after tracer administration was not significant. We compared the tumor-bearing and tumor-naïve animals to show that ⁶⁴Cu-NODAGA-PEG4-SL022-GGS was retained in the marrow rich skeletal sites of tumor-bearing compared to tumor-naïve animals (FIG. 4C and Table 6). As expected, the bones of tumor-bearing animals retained significantly more ⁶⁴Cu-NODAGA-PEG4-SL022-GGS than the tumor-naïve controls (6.9±1.03% ID/g versus 0.1±0.06% ID/g, P=0.00003). The in vivo and ex vivo biodistribution data together demonstrated that ⁶⁴Cu-NODAGA-PEG4-SL022-GGS accumulated in the marrow rich bones within minutes of injection and cleared through the kidneys slower than it did from the non-target organs. The retention of ⁶⁴Cu-NODAGA-PEG4-SL022-GGS in target organs was significantly higher than the retention in non-target organs at 1 and 4 h after tracer administration and did not accumulate in the marrow rich bones of tumor naïve animals.

TABLE 6
biodistribution of ⁶⁴Cu-NODAGA-PEG4-SL022-GGS in tumor-bearing
mice and tumor-naïve mice at 4 hour timepoint

| | |
|---|---|
| Tumor bearing | Tumor naïve |
| (N = 4) | (N = 4) |

| | % ID/g | SD | % ID/g | SD |
|---|---|---|---|---|
| Blood | 0.03 | 0.01 | 0.02 | 0.01 |
| Lung | 0.15 | 0.04 | 0.07 | 0.01 |
| Liver | 0.50 | 0.14 | 0.20 | 0.02 |
| Spleen | 0.08 | 0.03 | 0.06 | 0.01 |
| kidney | 2.60 | 0.67 | 3.89 | 0.53 |
| bladder | 1.21 | 1.20 | 3.67 | 0.97 |
| Muscle | 0.01 | 0.00 | 0.02 | 0.01 |
| Fat | 0.01 | 0.01 | 0.01 | 0.00 |
| Heart | 0.04 | 0.01 | 0.02 | 0.00 |
| Brain | 0.01 | 0.00 | 0.01 | 0.00 |
| Small intestine | 0.29 | 0.17 | 0.11 | 0.04 |
| U large intestine | 0.50 | 0.20 | 0.16 | 0.04 |
| L Large | 0.64 | 0.49 | 0.18 | 0.09 |
| intestine | | | | |
| Bone | 6.91 | 1.03 | 0.07 | 0.05 |

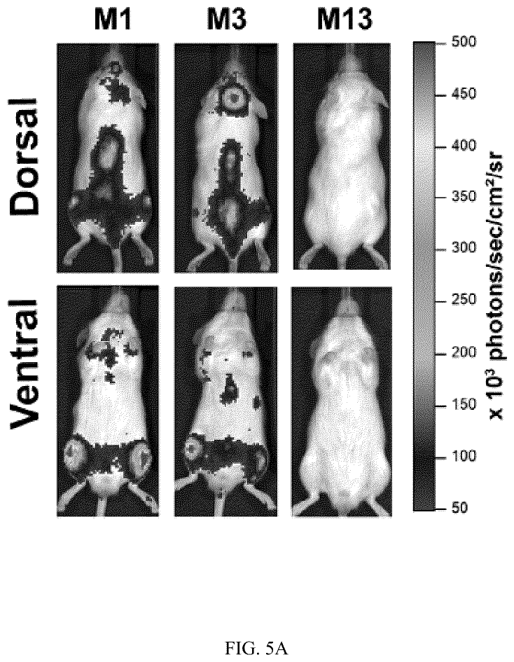

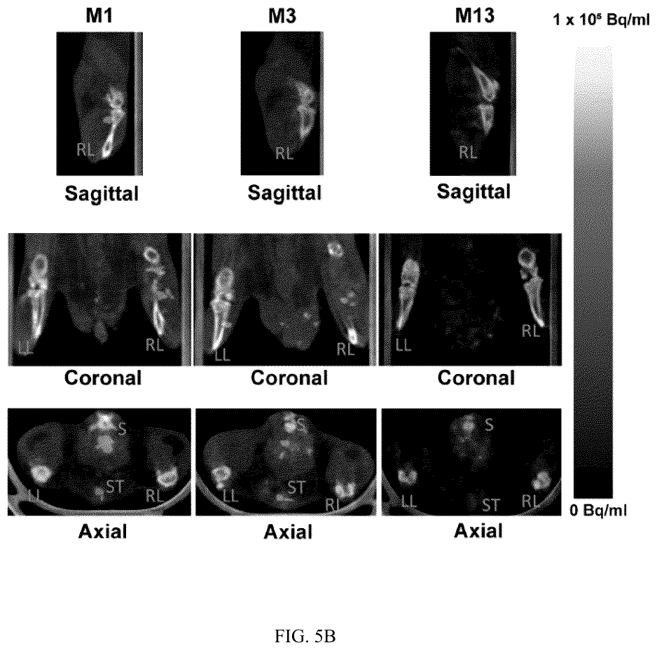

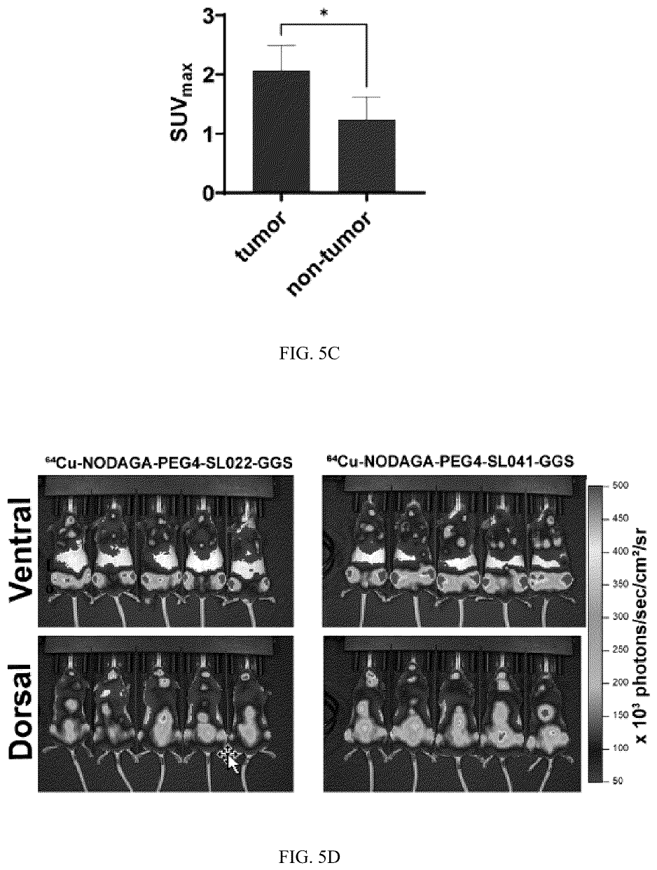

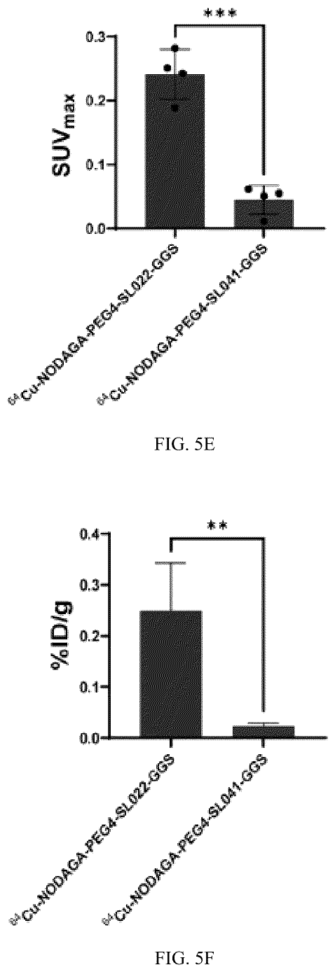

Next, we investigated the behavior of ⁶⁴Cu-NODAGA-PEG4-SL022-GGS in a disseminated tumor model to better understand the tracer's performance against background accumulation in tissues expressing the endogenous target receptor. The disseminated MM model was generated in immunocompromised animals through intravenous implantation of MM.1S-CBR-GFP-WT human MM cells (N=10). The disease progression was monitored by imaging the bioluminescent signal from the CBR reporter in MM.1S-CBR-GFP-WT cells (FIG. 5A). After establishing the diseased state, the animals were imaged with ⁶⁴Cu-NODAGA-PEG4-SL022-GGS/PET 1 h after tracer administration. Representative PET images in FIG. 5B showed that ⁶⁴Cu-NODAGA-PEG4-SL022-GGS accumulated in marrow rich bones (femur, tibia, spine, and pelvis) of tumor-bearing animals. The data in FIG. 5C showed the maximum standard uptake values (SUVₘₐₓ) quantified in the bones demonstrated the tracer accumulation was significantly higher when compared to the tumor naïve animals (N=5) (SUV 2.06±0.4 versus 1.24±0.4, P=0.02). ⁶⁴Cu-NODAGA-PEG4-SL022-GGS accumulation in the bones of tumor-naïve animals was expected and was likely due to the normal expression of CD38 by the cells of hematopoietic lineage present in the bone marrow. To further illustrate the in vivo specificity of ⁶⁴Cu-NODAGA-PEG4-SL022-GGS, we compared its target organ accumulation to that of the non-specific “scrambled” peptide, ⁶⁴Cu-NODAGA-PEG4-SL041-GGS (SL041: Thr-Tyr-His-Ile-Pro-Ile-Val; SEQ ID NO:29) (Table 4). We implanted MM.1S-CBR-GFP-WT MM cells in NSG mice (N=10). After the tumors were established (FIG. 5D), animals were injected with either ⁶⁴Cu-NODAGA-PEG4-SL022-GGS (N=5) or ⁶⁴Cu-NODAGA-PEG4-SL041-GGS (N=5) at 6.6 MBq/nM per animal. PET was performed 2 h after the tracers' administration. The data in FIG. 5E and FIG. 12 demonstrated significant ⁶⁴Cu-NODAGA-PEG4-SL022-GGS accumulation in leg bones of the animals compared to that of the non-specific ⁶⁴Cu-NODAGA-PEG4-SL041-GGS peptide (SUVₘₐₓ 0.24±0.04 versus SUVₘₐₓ 0.05±0.02, P=0.0001). When the leg bones were excised after the completion of PET and accessed for the associated activity, the ⁶⁴Cu-NODAGA-PEG4-SL022-GGS showed significantly higher accumulation compared to the non-specific peptide (FIG. 5F, 0.25±0.09% ID/g versus 0.02±0.006% ID/g, P=0.003). Overall, PET data demonstrated the in vivo validity of ⁶⁴Cu-NODAGA-PEG4-SL022-GGS as a CD38-specific PET imaging agent.

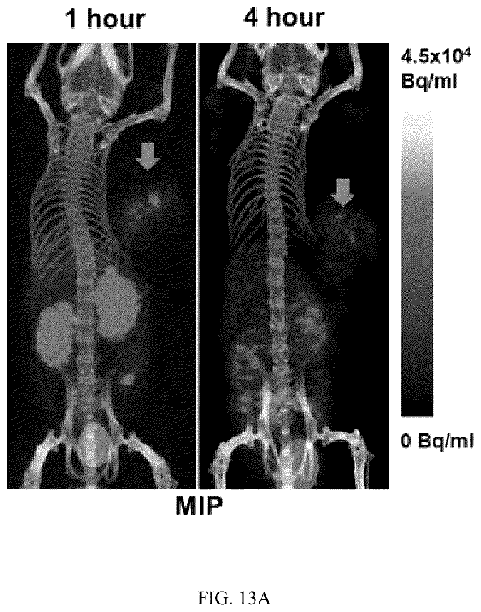

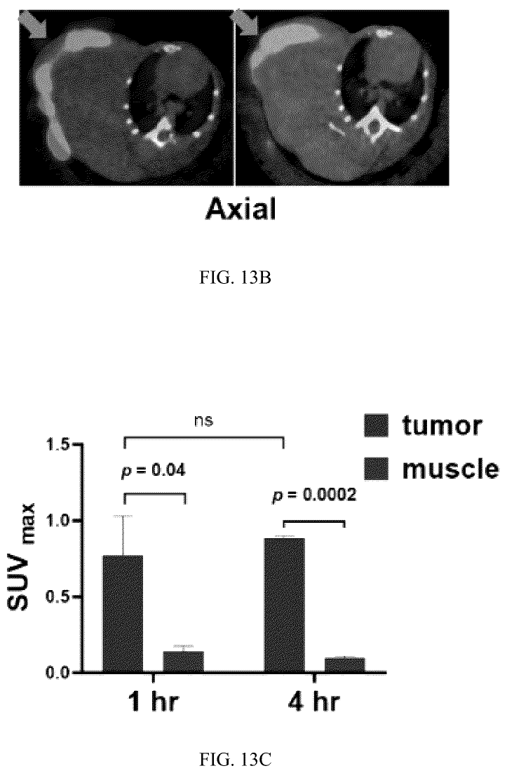

We further investigated the performance of ⁶⁴Cu-NODAGA-PEG4-SL022-GGS as an imaging agent in a subcutaneous model of MM. Subcutaneous MM.1S-CBR-GFP-WT xenografts were generated in immunocompromised animals (N=3). After the tumors reached 200 mm³ (approximately), the animals were imaged with ⁶⁴Cu-NODAGA-PEG4-SL022-GGS at 1 h and 4 h after administration. The representative images in FIGS. 13A and 13B showed that ⁶⁴Cu-NODAGA-PEG4-SL022-GGS accumulated and was retained in tumors for 4 h. SUVₘₐₓ were significantly higher than background (muscle) at both time points, (0.8±0.3 for bone and 0.1±0.04 muscle, P=0.04 at 1 h and 0.9±0.01 for bone and 0.1±0.01 for muscle, P=0.0002 at 4 h) (FIG. 13C). As anticipated, clearance through kidneys decreased at 4 h after tracer administration. The difference in ⁶⁴Cu-NODAGA-PEG4-SL022-GGS retention in tumors at both time points was not significant, which suggested that the compound remained in tumors despite the kidney clearance.

What is claimed is:

1. An imaging agent composition, comprising a CD38 targeting peptide covalently attached to or complexed with a radionuclide binding structure.

2. The composition of claim 1, wherein the radionuclide binding structure comprises a chelator.

3. The composition of claim 2, wherein the chelator comprises one of 1,4,8,11-tetraazacyclotetradecane-1-(methane phosphonic acid)-8-(methane carboxylic acid) (TE1A1P), dodecane tetraacetic acid (DOTA), diethylenetriaminepentaacetic acid (DTPA), 1,4,8, 11-tetraazacyclotetradecane-1-(methane phosphonic acid)-8-(methane carboxylic acid) (CB-TE1A1P), 1,4,7-triazacyclononane, 1-glutaric acid-4,7-acetic acid (NODAGA), and 1,8-diamino-3,6,10,13, 16, 19-hexaazabicyclo(6,6,6) eicosane (diamine-sarcophagine) (Diamsar).

4. The composition of claim 1, wherein the CD38 targeting peptide comprises an amino acid sequence selected from THYPIVI (SEQ ID NO:1), FSRDWTS, (SEQ ID NO:2), VNYHFPV (SEQ ID NO:3), TPYPIVL (SEQ ID NO: 4), YDWTMHS (SEQ ID NO:5), VSYHFPV (SEQ ID NO:6), YKDWSEW (SEQ ID NO:7), DLVHYPE (SEQ ID NO:8), MPVARDW (SEQ ID NO:9), QGTDWTM (SEQ ID NO:10), QTSHDWL (SEQ ID NO:11), HAPWFRGG (SEQ ID NO: 12), THYPIVIGGSK (SEQ ID NO:13), FSRDWTSGGSK (SEQ ID NO:14), TPYPIVLGGSK (SEQ ID NO:15), YDWTMHSGGSK (SEQ ID NO:16), VSYHFPVGGSK (SEQ ID NO:17), YKDWSEWGGSK (SEQ ID NO:18), HAPWFRGGGGS (SEQ ID NO: 19), VIVYHFPVGGSK (SEQ ID NO:20), THYPIVIGGS (SEQ ID NO:21), FSRDWTSGGS (SEQ ID NO:22), TPYPIVLGGS (SEQ ID NO:23), YDWTMHSGGS (SEQ ID NO:24), VSYHFPVGGS (SEQ ID NO: 25), YKDWSEWGGS (SEQ ID NO:26), HAPWFRGGGG (SEQ ID NO:27), and VIVYHFPVGGS (SEQ ID NO:28).

5. The composition of claim 1, wherein the radionuclide comprises a radiometal.

6. The composition of claim 5, wherein the radiometal comprises one of ⁶⁴Cu, ⁶⁷Cu, ²¹²Pb, ¹¹¹In, and ⁶⁸Ga.

7. The composition of claim 1, further comprising a linker covalently attached to or complexed with the CD38 targeting peptide and the radionuclide binding structure, wherein the linker is configured to attach the CD38 targeting peptide to the radionuclide binding structure.

8. The composition of claim 1, wherein the imaging agent comprises one of ⁶⁴Cu-NODAGA-PEG4-THYPIVIGGSK, ⁶⁴Cu-NODAGA-PEG4-FSRDWTSGGSK, ⁶⁴Cu-NODAGA-PEG4-TPYPIVLGGSK, ⁶⁴Cu-NODAGA-PEG4-YDWTMHSGGSK, ⁶⁴Cu-NODAGA-PEG4-VSYHFPVGGSK, ⁶⁴Cu-NODAGA-PEG4-YKDWSEWGGSK, and ⁶⁴Cu-NODAGA-PEG4-HAPWFRGGGGS.

9. The composition of claim 1, wherein the imaging agent comprises one of ⁶⁴Cu-NODAGA-PEG4-THYPIVIGGS, ⁶⁴Cu-NODAGA-PEG4-FSRDWTSGGS, ⁶⁴Cu-NODAGA-PEG4-TPYPIVLGGS, ⁶⁴Cu-NODAGA-PEG4-YDWTMHSGGS, ⁶⁴Cu-NODAGA-PEG4-VSYHFPVGGS, and ⁶⁴Cu-NODAGA-PEG4-YKDWSEWGGS.

10. The composition of claim 1, wherein the imaging agent is ⁶⁴Cu-NODAGA-PEG4-THYPIVIGGS.
